(19) United States
(12) Patent Application Publication
(10) Pub. No.: US 2005/0141729 A1
(43) Pub. Date: Jun. 30, 2005

(54) Ear-attaching type electronic device and biological information measuring method in ear-attaching type electronic device

(75) Inventors: Takashi Kanzaki, Tokyo (JP); Masazumi Niimi, Tokyo (JP)

Correspondence Address: FRISHAUF, HOLTZ, GOODMAN & CHICK, PC, 767 THIRD AVENUE, 25TH FLOOR, NEW YORK, NY, 10017-2023, US

(73) Assignee: Casio Computer Co., Ltd., Tokyo (JP)

(21) Appl. No.: 11/018,140

(22) Filed: Dec. 20, 2004

(30) Foreign Application Priority Data
Dec. 26, 2003 (JP) 2003-434069
Dec. 26, 2003 (JP) 2003-434881
Oct. 8, 2004 (JP) 2004-295995

(51) Int. Cl. A61B 7/04; A61B 5/02

(52) U.S. Cl. 381/67; 600/528

(57) ABSTRACT

An ear-attaching type electronic device includes: a body part supported in a vicinity of a lower part of an occipital part when the device is attached; a pair of arm parts extending from the body part, to which a connecting member is placed therein; and a pulse sensor section for detecting pulse by being attached to an earlobe, wherein advice is outputted according to a comparison between a detected pulse rate and a previously-set pulse rate range.

### CROSS-REFERENCE TO RELATED APPLICATIONS

This application is based upon and claims the benefit of priority from the prior Japanese Patent Application No. 2003-434069, filed Dec. 26, 2003, and 2003-434881, filed Dec. 26, 2003, and 2004-295995, filed Oct. 08, 2004, and the entire contents of which are incorporated herein by reference.

### BACKGROUND OF THE INVENTION

1. Field of the Invention

The present invention relates to an ear-attaching type electronic device and a biological information measuring method in the ear-attaching type electronic device, the ear-attaching electronic device being structured to be capable of measuring biological information and outputting sound simultaneously.

2. Description of Related Art

Conventionally, a measuring device which measures biological information regarding bloodstream of a human body, such as pulse, heart rate and the like, is known. Such an measuring device is not only used as a medical device, but is also widely common for home use in order to maintain health and to recognize an exercise condition. Further, a product of these electronic devices takes various shapes and sizes. For example, there is an electronic device product which is downsized to be portable, or an electronic device product integrally incorporated within another product.

Concretely, the measuring device measures pulse or heart rate of a user when the user is doing an exercise such as walking, jogging or the like. Then, the measuring device sets an interval of pitch sound according to measured pulse or heart rate so as to achieve exercise amount which is appropriate to a purpose of the exercise that the user is doing, and the measuring device outputs the pitch sound based on the set interval. The user does the walking or jogging based on the pitch sound outputted from the measuring device, and thereby it is possible to maintain appropriate pace.

Here, as a method to measure pulse by the measuring device, there is a method which measures pulse by contacting a finger to a pulse sensor provided in a wristwatch. However, with this method, it is necessary to contact a finger to the wristwatch at each time of measuring pulse, and therefore it is difficult to occasionally measure pulse during the exercise.

In addition, what is available is a headphone type, measuring device which comprises a belt on which ECG (electrocardiogram) measuring electrodes are placed and a headphone, wherein the belt is wound up on a body such as chest, abdomen or the like, and heart rate measured through the ECG measuring electrodes is outputted from the headphone with sound.

However, with this measuring device, a cable which connects between the belt wound up on the body and the headphone is in a state of being suspended from a head to the body. Therefore, the cable floats during exercise, whereby, it bothers the exercise, for example, it bothers a swinging arm at the time of jogging.

Further, since a posture of the headphone at the time of attachment is maintained only according to elasticity corresponding to the bending of an arm part, there is the case that the arm part goes out of alignment or gets disengaged easily due to the movement of user's body, especially the movement of a head part.

Further, in order to measure heart rate, it is necessary to wind the belt up on the body. Accordingly, its attaching operation and sense of the attachment are bothersome.

Further, in order to enjoy walking or the like, the case that a user is walking or the like while listening to the music or the radio is assumed. In this case, it is necessary to carry a music playing device, a portable radio or the like, in addition to the measuring device for measuring pulse or heart rate. Therefore, it is extremely inconvenient.

Further, the measuring device for measuring pulse or heart rate is a different type of device from a music playing device, a portable radio or the like. Therefore, while listening to the music or the radio, it is not possible to hear pitch sound from the measuring device. To the contrary, while hearing pitch sound from the measuring device, it is not possible to listen to the music or the radio. Therefore, it is extremely inconvenient.

In addition, as one example of such a headphone type measuring device, there is a measuring device in which an arm part connects between a headphone and a body part comprising a sound outputting unit for outputting a sound signal, a code is placed for electrically connecting the headphone and the body part inside of the arm part, and the arm part is structured to be rotatable with respect to the body part so as to fold the arm part to be held.

However, if such a headphone type measuring device is folded to be housed, the code gets wrenched according to the rotation of the arm part with respect to the body part, and there is a possibility of breaking the code.

### SUMMARY OF THE INVENTION

An object of the present invention is to provide an ear-attaching type electronic device and a biological information measuring method in the ear-attaching type electronic device, being capable of measuring biological information while enjoying music, wherein each of left and right arm parts which protrude from a body part which is maintained around an occipital area when the device is attached and an electric connecting member is placed, is structured to be rotatable with respect to the body part.

### BRIEF DESCRIPTION OF THE DRAWINGS

The present invention will become more fully understood from the detailed description given hereinafter and the accompanying drawing given by way of illustration only, and thus are not intended as a definition of the limits of the present invention, and wherein:

FIG. 1A is a perspective view showing an ear-attaching type device in the first embodiment of the present invention,

FIG. 1B is a view showing an attachment state of the ear-attaching type device in the first embodiment of the present invention,

FIG. 2A is a front view of the ear-attaching type device in the first embodiment of the present invention,

FIG. 2B is a rear view showing the ear-attaching type device in the first embodiment of the present invention,

FIG. 2C is a view showing a displaying unit of the ear-attaching type device in the first embodiment of the present invention,

FIG. 3A is a right side view showing the ear-attaching type device in the first embodiment of the present invention,

FIG. 3B is a left side view showing the ear-attaching type device in the first embodiment of the present invention,

FIG. 4 is a partly-omitted sectional view taken along the IV-IV line of FIG. 2A,

FIG. 5 is a partly-omitted sectional view showing a device housing state of the ear-attaching type device in the first embodiment of the present invention,

FIG. 6 is a view showing a left arm of the ear-attaching type device in the first embodiment of the present invention,

FIG. 7 is a bottom view showing the left arm of the ear-attaching type device in the first embodiment of the present invention,

FIG. 8 is a sectional view taken along the VIII-VIII line of FIG. 6,

FIG. 9 is a sectional view taken along the IX-IX line FIG. 6,

FIG. 10 is a perspective view showing a panel-side case of the ear-attaching type device in the first embodiment of the present invention,

FIG. 11 is a perspective view showing a power-side case of the ear-attaching type device in the first embodiment of the present invention,

FIG. 12 is a sectional view taken along the XII-XII line of FIG. 2B,

FIG. 13A is a block diagram showing an internal structure of the ear-attaching type device in the first embodiment of the present invention,

FIG. 13B is a view showing a data structure accessed in the RAM 104 in the first embodiment of the present invention,

FIG. 13C is a view showing a structure of data and programs stored in the ROM 102 in the first embodiment of the present invention,

FIG. 14A is a view showing a data structure of an exercise purpose table in the first embodiment of the present invention,

FIG. 14B is a view showing a data structure of an advise sound storing area in the first embodiment of the present invention,

FIG. 15A is a view showing a pulse rate accumulation storing area in the first embodiment of the present invention,

FIG. 15B is a view showing an individual data in the first embodiment of the present invention,

FIG. 15C is a view showing a set range data in the first embodiment of the present invention,

FIG. 16 is a view describing a method to calculate a pulse rate in the first embodiment of the present invention,

FIG. 17 is a view describing exercise intensity in the first embodiment of the present invention,

FIG. 18 is a flowchart illustrating an operation of an device controlling process in the first embodiment of the present invention,

FIG. 19 is a flowchart illustrating an operation of a first pulse measuring process in the first embodiment of the present invention,

FIG. 20 is a flowchart illustrating an operation of a sound reporting process in the first embodiment of the present invention,

FIG. 21A is a flowchart illustrating an operation of an interruption reporting process in the first embodiment of the present invention,

FIG. 21B is a view describing the operation of the interruption reporting process in the first embodiment of the present invention,

FIG. 22A is a view showing a state transition of the ear-attaching type device on a display in the first embodiment of the present invention,

FIG. 22B is a graph showing a transition of pulse rate in the first embodiment of the present invention,

FIG. 23A is a block diagram showing an internal structure of an ear-attaching type device in the second embodiment of the present invention,

FIG. 23B is a view showing a data structure accessed in the RAM 104 in the second embodiment of the present invention,

FIG. 23C is a view showing a structure of data and programs stored in the ROM 102 in the second embodiment of the present invention,

FIG. 24A is a view showing a pitch time table in the second embodiment of the present invention,

FIG. 24B is a reporting range setting data in the second embodiment of the present invention,

FIG. 25 is a flowchart illustrating an operation of a second pulse measuring process in the second embodiment of the present invention,

FIG. 26 is a flowchart illustrating the operation of the second pulse measuring process in the second embodiment of the present invention,

FIG. 27 is a flowchart illustrating an operation of a first interval setting process in the second embodiment of the present invention,

FIG. 28 is a flowchart illustrating an operation of a second interval setting process in the second embodiment of the present invention,

FIG. 29 is a graph showing a transition of a pulse rate in the second embodiment of the present invention,

FIG. 30 is a magnified view showing a right arm supporting member for describing a biasing mechanism provided in the ear-attaching type device of the present invention,

FIG. 31A is a view showing an alternative of the ear-attaching type device of the present invention, and

FIG. 31B is a view showing an alternative of the ear-attaching type device of the present invention.

### PREFERRED EMBODIMENTS OF THE INVENTION

Hereinafter, a concrete embodiment will be described with reference to figures. However, the scope of the invention is not limited to illustrated figures.

### FIRST EMBODIMENT

[1-1 External Structure]

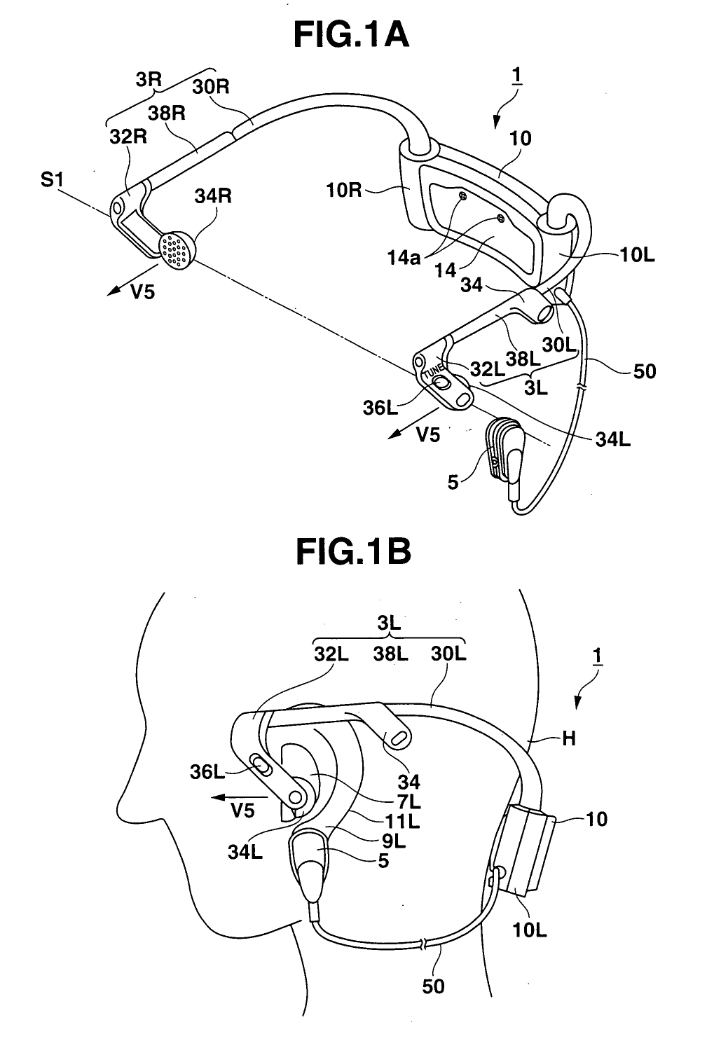

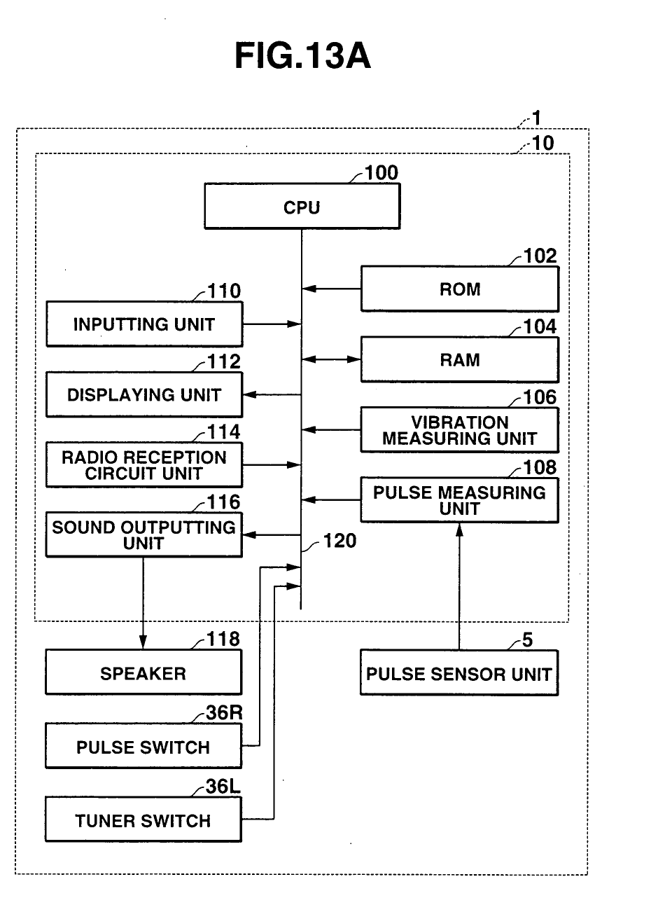

Hereinafter, a first embodiment of the case that an ear-attaching type electronic device of the present invention is applied to an ear-attaching type pulse measuring device (hereafter, it is referred to as “ear-attaching type device”) 1 will be described with reference from FIG. 1A to FIG. 13.

Here, directions under the description are assumed to be the directions with respect to a user who attaches the ear-attaching type device 1 to himself/herself. Concretely, it is assumed that a face side when attaching the ear-attaching type device 1 (toward left in FIG. 1B) is front, an occipital side (toward right in FIG. 1B) is back, a left ear side is left, a right ear side is right, an up side is up, and a down side is down. Further, it is assumed that a direction in which left and right arm parts 3R and 3L are facing, that is, a direction toward the center of a head part is an internal direction, and its opposite direction is an external direction.

First, an outline of the ear-attaching type device 1 will be described.

As shown in FIGS. 1A and 1B, the ear-attaching type device 1 comprises a body part 10, a right arm part 3R, a left arm part 3L, a right driver unit 34R and a left driver unit 34L each of which is a speaker unit, and a pulse sensor unit 5. The right arm part 3R is supported at the right upper end of the body part 10 so as to cause a bias in the internal direction.

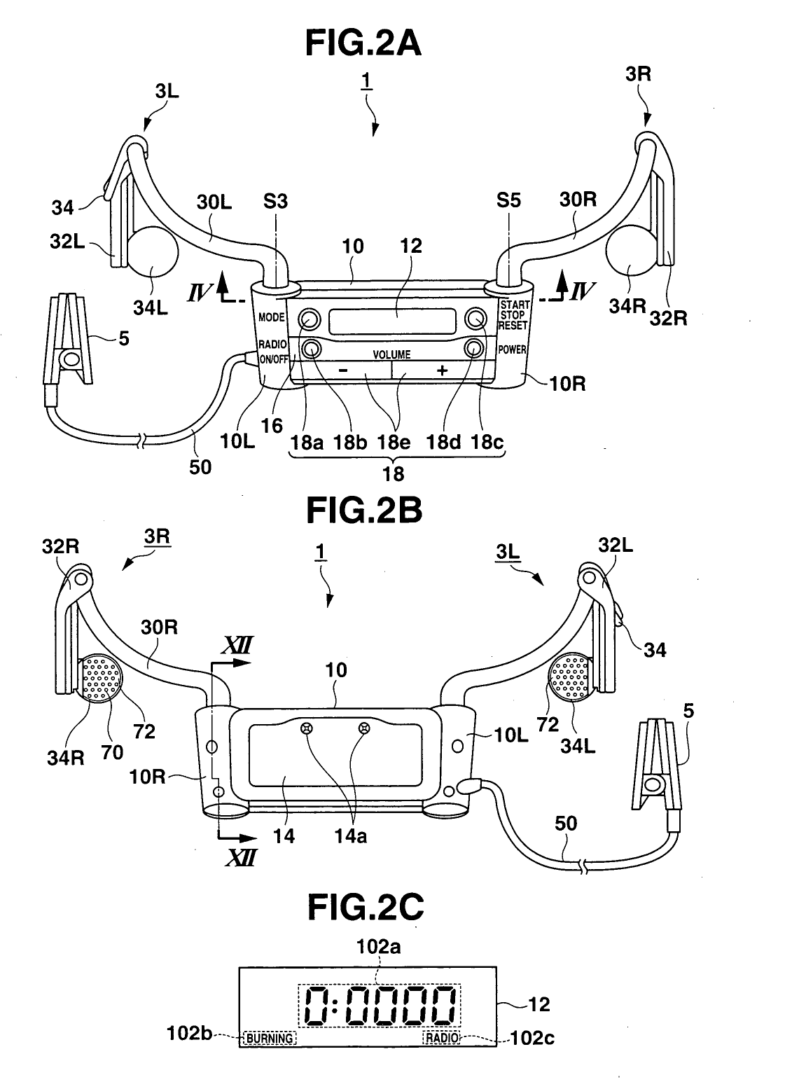

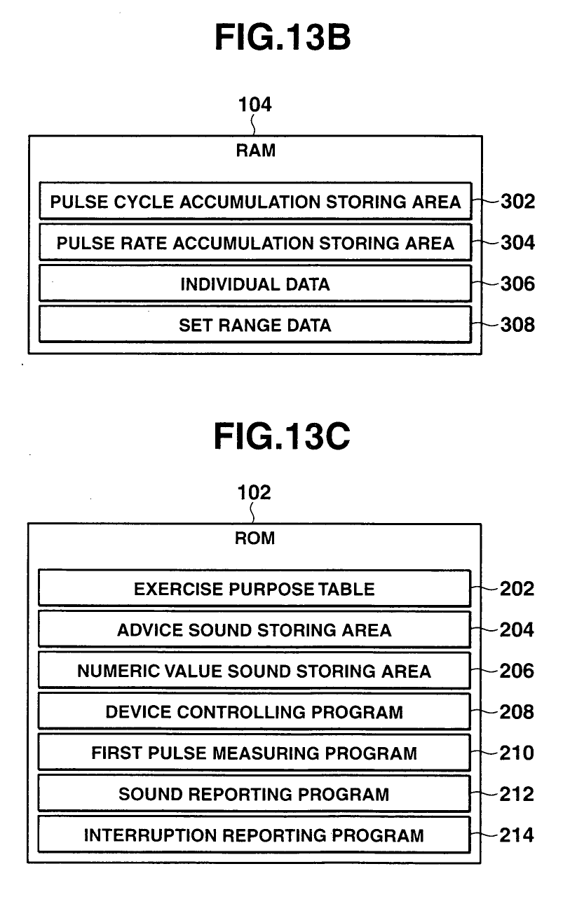

Further, as shown in FIGS. 2A and 2B, within the body part 10, incorporated are various control circuits, a power unit and the like, such as a radio reception circuit unit 114 (see FIG. 13A), a pulse measuring unit 108 (see FIG. 13A), a sound outputting unit 116 (see FIG. 13A). Further, an operating panel 16 is placed at the front of the body part 10. Further, a detachable lid 14 is formed on the back of the body part 10, and a right arm supporting member 1OR and a left arm supporting member 10L are formed at the right end and the left end of the body part 10, respectively.

On the operating panel 16, for example, a screen display 12 comprising an LCD (Liquid Crystal Display) or the like, and a various switch group 18 are placed.

FIG. 2C is a view showing one example of the screen display 12. The screen display 12 comprises a time displaying area 102a for displaying a time period for which pulse is being measured, an exercise purpose displaying area 102b for displaying a current exercise purpose, and a radio operation state displaying area 102c for indicating ON/OFF of the radio.

The various switch group 18 comprises a mode switch 18a for setting an operation mode of the ear-attaching type device 1, a radio switch 18b for starting reception of the radio, a start-stop switch 18c for starting or stopping an operation of a stopwatch and a pulse detection operation by the pulse sensor unit 5 simultaneously, a power switch 18d for turning ON/OFF the power, and a volume switch 18e for changing sound volume.

The lid 14 is detachably formed from the body part 10 with a screw 14a. With this lid 14 taken off, a battery change of the power unit is performed.

The right arm supporting member 10R is a mechanism for supporting the right arm part 3R, and the left arm supporting member 10L is a mechanism for supporting the left arm part 3L.

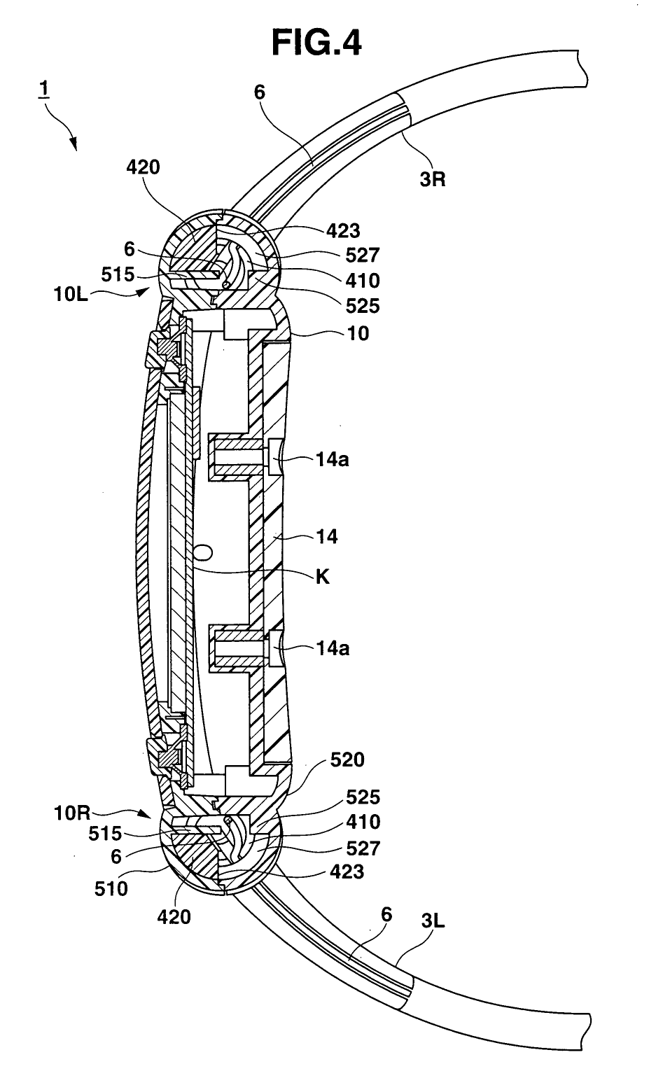

Inside of the right arm part 3R, a connecting member 6 (see FIG. 4) such as a connecting code or the like, for electrically connecting the right driver unit 34R and the sound outputting unit 116 of the body part 10 is placed. The right arm part 3R comprises a right arm 30R, a right connecting member 38R and a right driver unit supporting member 32R, wherein the right arm 30R, the right connecting member 38R and the right driver unit supporting member 32R are integrally formed.

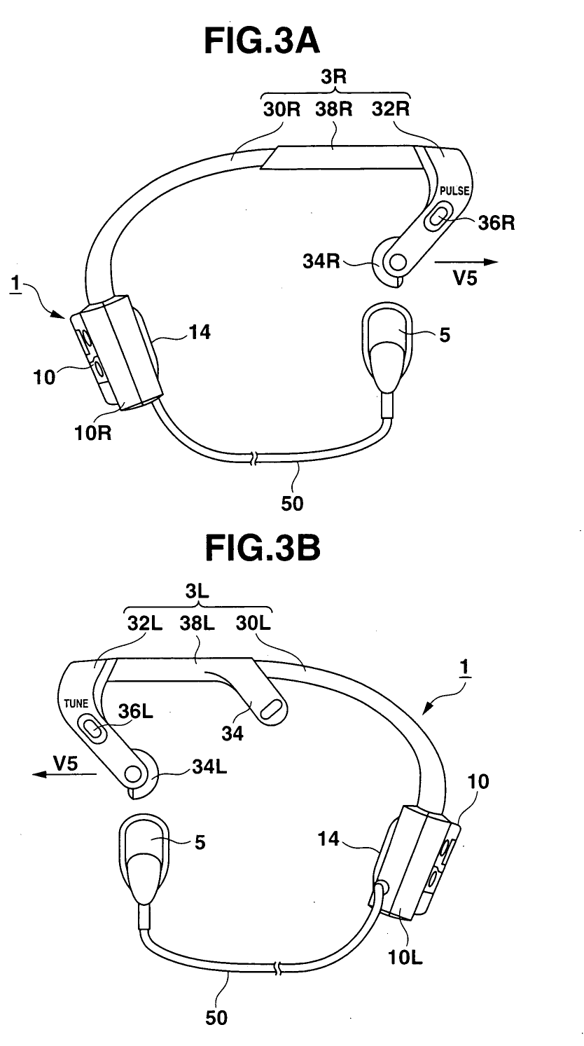

As shown in FIGS. 2A and 3A, the right arm 30R is formed so as to extend in the up direction from the upper end of the right arm supporting member 10R, and then to curve diagonally in the right-up-back direction, to form approximately a half circle in the external direction after all.

The right connecting member 38R is for bridging between the right arm 30R and the right driver unit supporting member 32R. Concretely, the right connecting member 38R is, for example, formed in a cylindrical shape, and one edge of the right arm 30R is inserted into the cylinder of the right connecting member 38R to be fixed, and another edge of the right connecting member 38R supports the right driver unit supporting member 32R in a down direction, for bridging.

Here, it is also possible to structure the right connecting member 38R and the right arm 30R not to be fixed but to be stretchable for adjusting the full length of the right arm part 3R.

The right driver unit supporting member 32R is formed in a plate shape and is used for supporting the right driver unit 34R. Concretely, as shown in FIG. 3A, the right driver unit supporting member 32R is formed in approximately a letter of ‘L’ so as to extend diagonally in the back-down direction, and the right driver unit 34R is supported at the edge part thereof. Further, on the right side surface of the right driver unit supporting member 32R, a pulse switch 36R for outputting sound which announces a pulse rate right after the measurement is placed.

With the right arm part 3R which comprises the right arm 30R, the right connecting member 38R and the right driver unit supporting member 32R, an arm curved along a temporal shape of a general human body from an ear hole 7R (illustration omitted) to an occipital part H is formed.

Here, described is the case that each of the arm parts 3R and 3L is formed in a curved shape. However, the present invention is not limited to the curved shape, and it is possible to form each of the arm parts 3R and 3L so as to bend the arm parts in a linear fashion so that it looks like a letter of ‘L’ shape when it is seen from the top view.

The right driver unit 34R is a speaker unit. Further, the right driver unit 34R is formed in approximately a half sphere shape so as to be insertable into the ear hole 7R, and the speaker 118 is placed inside thereof. At the bottom surface of the half sphere, a sound emitting surface 72 on which a plurality of holes 70 for emitting sound are created is provided. Further, a side part of the half sphere of the right driver unit 34R is supported by the right driver unit supporting member 32R so as to direct the sound emitting surface 72 in the direction of an arrow V5, which is the front direction, when the ear-attaching type device 1 is attached. The connecting member 6 which connects the body part 10 and the speaker 118 is placed in the body part 10 through the inside of the right driver unit supporting member 32R, the right connecting member 38R and the right arm 30R.

Since the right driver unit 34R is formed in approximately a half sphere shape in this way, it is possible to create a certain friction with an ear hole when it is attached, whereby it is possible to obtain a certain sense of attachment and stability by only inserting it into the ear hole. Further, since the sound emitting surface 72 (bottom surface) is in approximately a half sphere shape and facing in the front direction, it is possible not to entirely shut an ear hole from outside, whereby sound from outside is not entirely blocked. Accordingly, for example, even in the case that sound is being outputted from the speaker 118 during jogging, it is possible to hear sound from outside in regard to traffic.

The left arm part 3L comprises a left arm 30L, a left connecting member 38L and a left driver unit supporting member 32L, wherein the left arm 30L, the left connecting member 32L and the left driver unit supporting member 32L are integrally formed.

Here, since the left arm part 3L has approximately the same structure as the right arm part 3R, a different part from the structure from the right arm part 3R will be described hereafter.

At the left side surface of the left driver unit supporting member 32L, a tuner switch 36L for tuning in the radio is placed.

Further, a flange 34 which is formed in a plate shape is connected to the rear side edge of the left connecting member 38L. The flange 34 is used for pinching and fixing the pulse sensor unit 5 while a user is not using the pulse sensor unit 5.

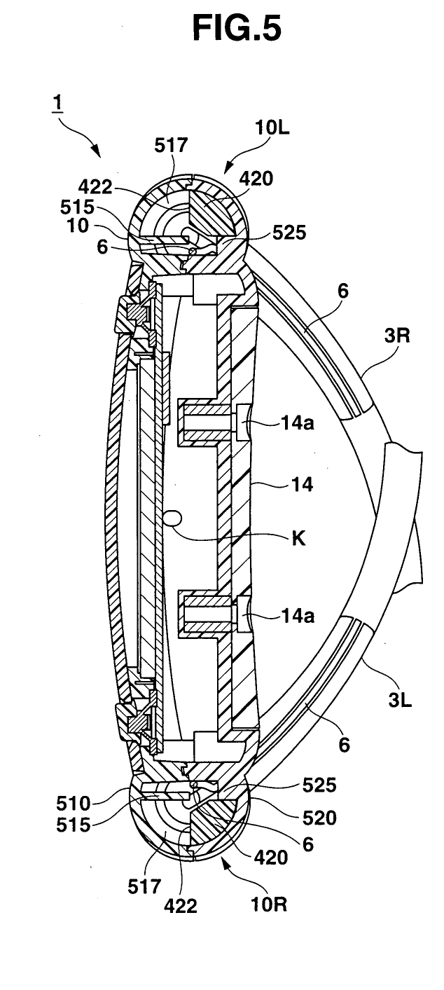

The right arm part 3R and the left arm part 3L having the above-described structure are rotatable with respect to the body unit 10, from a device attaching position at the time of attaching the ear-attaching type device 1 with a ear (see FIG. 4) to a device housing position at the time of housing the ear-attaching type device 1 being unused (see FIG. 5).

Hereinafter, a rotation mechanism of the right arm part 3R and the left arm part 3L will be described.

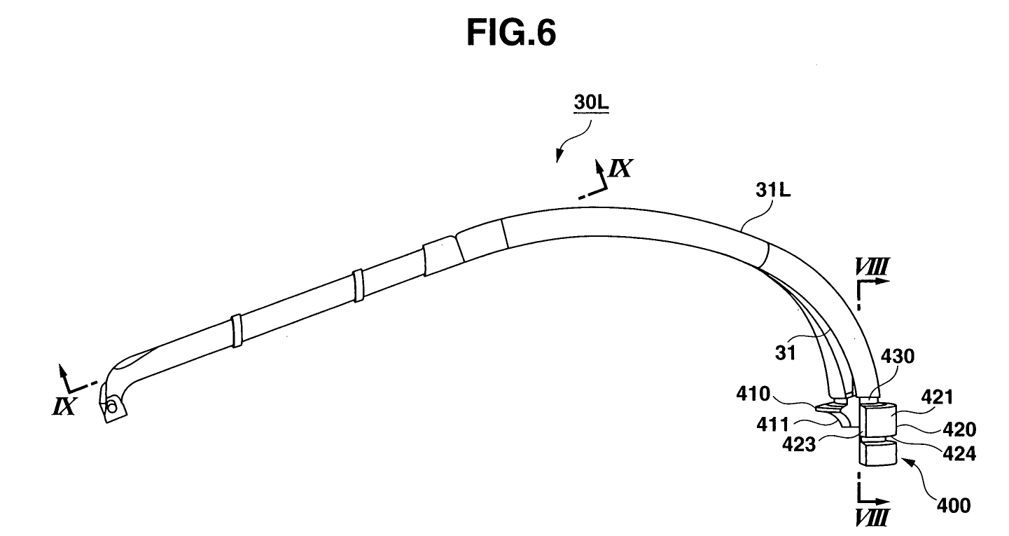

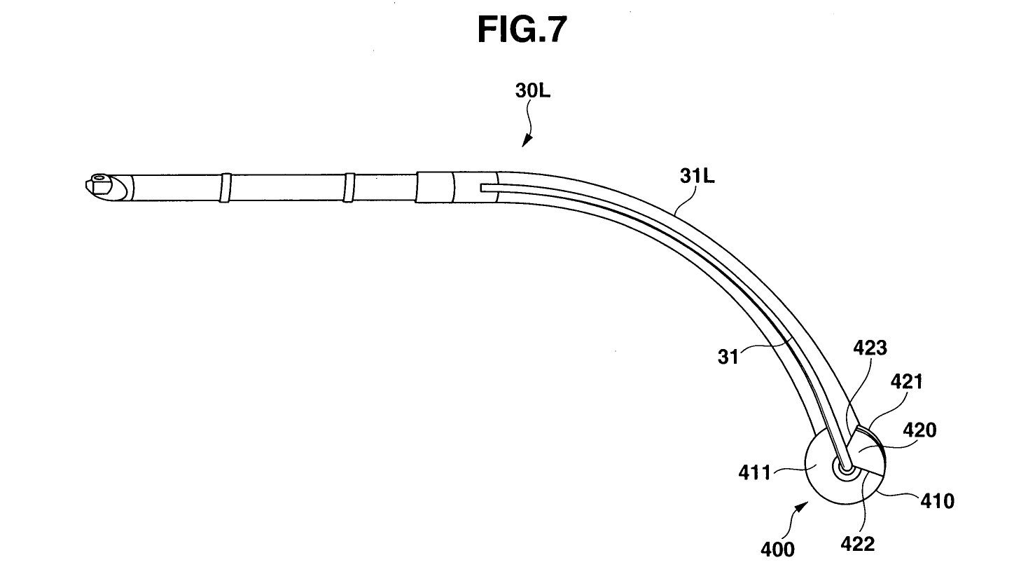

As shown in FIGS. 6 and 7, the left arm 30L of the left arm part 3L comprises a shaft member 400 which structures a rotation shaft S3 (see FIG. 2A) of the left arm 30L, at a position inside of the left arm supporting member 10L by being attached to the edge part of the side of the left arm supporting member 10L, that is, to the body part 10.

The shaft member 400 comprises a flange member 410 which has larger diameter than an arm body part 31L of the left arm 30L, the flange member 410 gradually becoming thicker as coming close to the rotation shaft S3, and a sliding guide 420 for guiding rotation of the left arm 30L so as to slide an external surface 421 thereof against an internal sliding surface 516 of a panel-side case 510 and an internal sliding surface 526 of a power-side case 520 (which will be described later) structuring the body part 10 together, wherein the flange member 410 and the sliding guide 420 are integrally formed.

Concretely, on the left arm 30L, a notch part 31 is formed by notching as much as a predetermined depth from the surface toward the center, from the arm body part 31L to the shaft member 400 to place the connecting member 6 therein. For example, as shown in FIG. 7, in the plane view, the flange member 410 and the sliding guide 420 are provided with respect to the notch part 31.

The flange member 410 is formed in approximately a sector shape when it is seen from the plane view so as to have a predetermined arc length continuously at one edge part with respect to the notch part 31 of the shaft member 400. Further, the flange member 410 is formed in a curved shape with a predetermined curvature so as to dent an external surface 411, which is concretely the bottom surface in FIG. 6, toward the S3 side and along the internal sliding surfaces 516 and 526 (inside wall) of the panel-side case 510 and the power-side case 520.

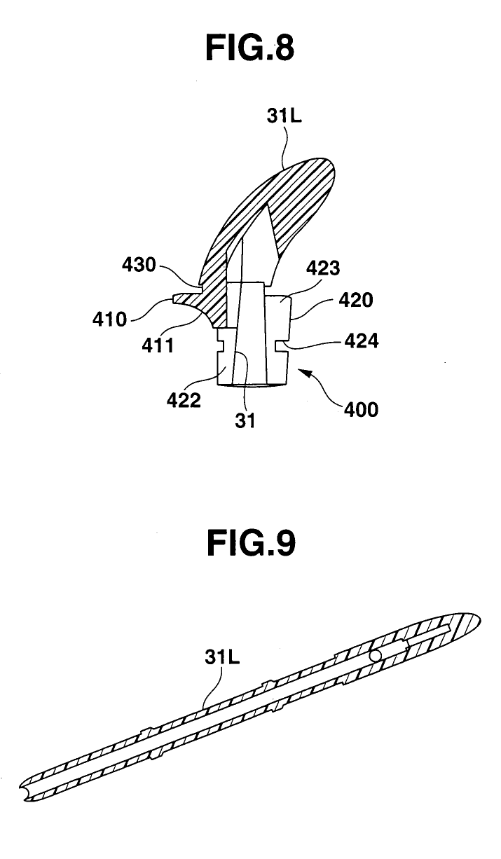

The sliding guide 420 is formed so as to form an external surface 421 thereof in approximately an arc shape with approximately the same radius as the flange member 410, and to connect the flange member 410 and another edge part of the notch part 31 of the shaft member 400. Further, the sliding guide 420 shares the edge surface at the arm body part 31L (for example, the upper edge surface in FIG. 6) with the flange member 410, and has certain amount of thickness in the shaft direction of the rotation shaft S3. Further, as shown in FIG. 8, the sliding guide 420 comprises a device position rotation stopping surface 422 and housing position rotation stopping surface 423 (rotation stopping surface) for stopping the rotation of the left arm part 3L at the device attaching position and the device housing position, respectively, so as to extend in the radial direction from the rotation shaft S3 and to continue to the external surface 421. More concretely, for example, the attaching position rotation stopping surface 422 and the housing position rotation stopping surface 423 are placed so as to make an angle of the two surfaces approximately orthogonal with the rotation shaft S3 defined as its vertex.

Further, on the external surface 421 of the sliding guide 420, a groove 424 which has approximately a rectangular shape when it is seen in a cross-sectional view is formed in the sliding direction to be engaged to a rib 517 which protrudes from the inside surface of the panel-side case 510 and the power-side case 520.

As described above, by the flange member 410 and the sliding guide 420 structuring the shaft member 400, a rotation mechanism portion for rotating the left arm part 3L with respect to the body part 10 is structured.

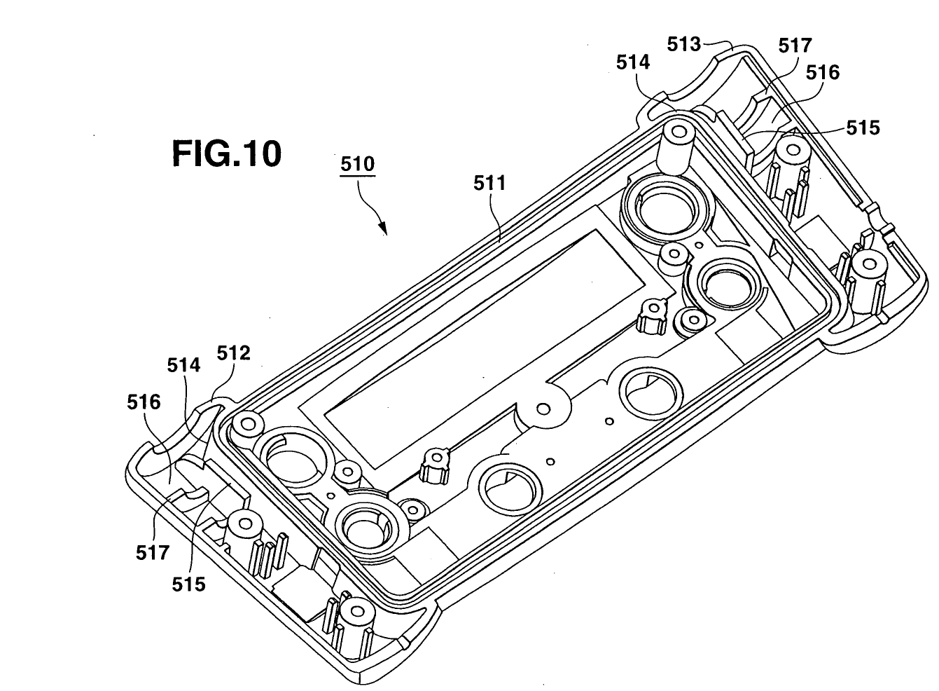

Here, as shown in FIG. 9, an edge part at the left connecting member 38L of the arm body part 31L is formed to have a cylindrical shape, and it is possible to place the connecting member 6 (illustration omitted) therein.

Further, since the right arm part 3R has approximately the same structure as the left arm part 3L, detailed description thereof is omitted.

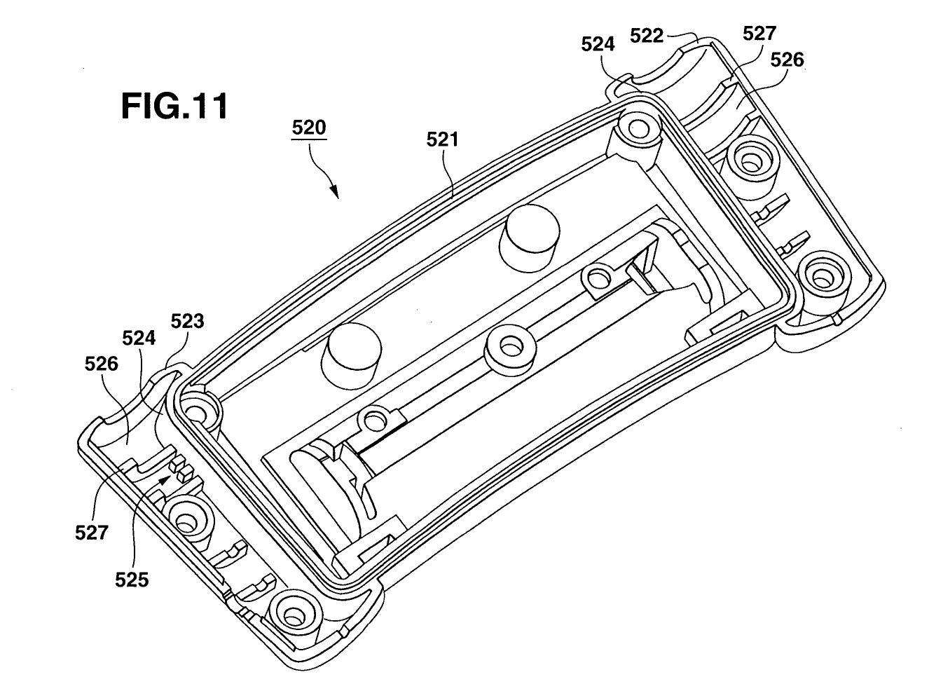

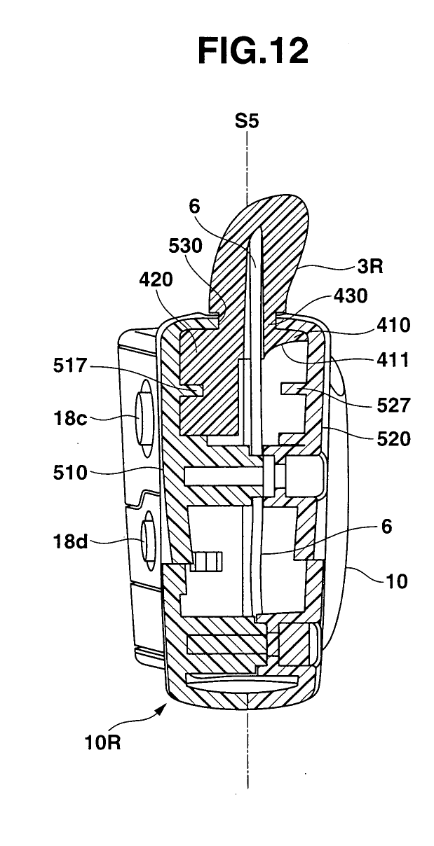

The body part 10 which comprises the right arm supporting member 10R and the left arm supporting member 10L, as shown in FIGS. 10 and 11, further comprises the panel-side case 510 (first body case member) and the power-side case 520 (second body case member), both of which are formed in a reentrant shape.

In other words, the panel-side case 510 and the power-side case 520 structure the right arm supporting member 10R and the left arm supporting member 10L by having both of the opening sides face each other.

The panel-side case 510 comprises a circuit board housing member 511 therein, in which a predetermined circuit board K (see FIG. 4) and the like are housed. Further, at both of edge parts with respect to this circuit board housing member 511, a right supporting member structuring portion 512 for structuring the right arm supporting member 10R and a left supporting member structuring portion 513 for structuring the left arm supporting member 10L are provided.

At each of the right supporting member structuring portion 512 and the left supporting member structuring portion 513, a panel-side internal wall portion 514 (internal wall) is formed in a curved shape so as to follow the external surface 411 of the flange member 410 of the rotating left arm part 3L and the right arm part 3R.

Further, at both the left and right edge sides of the panel-side internal wall portion 514, provided is a device position stopping portion 515 to which the attaching position rotation stopping surface 422 of the sliding guide 420 is to be contacted for stopping the rotation of the left and right arm parts 3R and 3L at the device attaching position. This attaching position stopping portion 515 is placed so as to protrude from inside of the right supporting member structuring portion 512 and the left supporting member structuring portion 513 toward the front side, with a small interval secured from the circuit board housing member 511. Thereby, it is possible to secure space for placing the connecting member 6 between the attaching position stopping portion 515 and the circuit board housing member 511.

Further, each of the right supporting member structuring portion 512 and the right supporting member structuring portion 513 comprises an internal sliding surface 516 which is formed so as to make curvature thereof approximately equal to the curvature of the external surface 421, which is a sliding surface of the sliding guide 420. At a predetermined position of the internal sliding surface 516, the rib 517 which is to be engaged with the groove 424 of the sliding guide 420 is provided so as to extend up to the edge part of the attaching position stopping portion 515 along the sliding direction.

In the power-side case 520, provided is a power arranging member 521 inside of which a predetermined battery and the like are arranged. Further, at both of left and right edge parts with respect to the power arranging member 521, a right supporting member structuring portion 522 and a left supporting member structuring portion 523 are placed for structuring the right arm supporting member 10R and the left arm supporting member 10L, respectively.

Each of the right supporting member structuring portion 522 and the left supporting member structuring portion 523 comprises a power-side internal wall portion 524 (internal wall) which is formed in a curved shape so as to follow the external surface 411 of the flange member 410 of the rotating left and right arm parts 3R and 3L.

Further, continuing from the internal surface of both the left and right edge sides of the power-side internal wall portion 524, placed is a housing position stopping portion 525 to which the housing position rotation stopping surface 423 of the sliding guide 420 is to be contacted to stop the rotation of the left and right arm parts 3R and 3L at the device housing position. This housing position stopping portion 525 is placed so as to protrude from the internal surface of the right supporting member structuring portion 522 and the left supporting member structuring portion 523 toward the front side.

Further, each of the right supporting member structuring portion 522 and the left supporting member structuring portion 523 comprises an internal sliding surface 526 which is formed so as to make curvature thereof approximately equal to the curvature of the external surface 421, which is a sliding surface of the sliding guide 420. At a predetermined position of the internal sliding surface 526, the rib 527 which is to be engaged with the groove 424 of the sliding guide 420 is placed so as to extend up to the edge part of the standing surface of the power arranging member 521 along the sliding direction.

According to the above-described structure, while the panel-side case 510 and the power-side case 520 are placed so as to face each other and the right arm part 3R and the left arm part 3L are respectively supported by the right arm supporting member 10R and the left arm supporting member 10L, in regard to the right arm part 3R and the left arm part 3L, it is possible to rotate the attaching position rotation stopping surface 422 of the sliding guide 420 until it is contacted with the attaching position stopping portion 515 of the panel-side case 510 and also possible to rotate the housing position rotation stopping surface 423 of the sliding guide 420 until it is contacted with the storing position stopping portion 525 of the power-side case 520.

In this way, by the attaching position stopping portion 515 of the panel-side case 510 and the housing position stopping portion 525 of the power-side case 520, a rotation stopping mechanism portion for stopping the rotation of the left and right arm parts 3R and 3L is structured.

Here, at the upper edge part of the right arm supporting member 10R and the left arm supporting member 10L, provided is an opening portion 530 for letting the left and right arm parts 3R and 3L, which are respectively attached to the right arm supporting member 10R and the left arm supporting member 10L, extend from the body part 10. This opening portion 530 is an opening having a smaller diameter than the arm body part 31L, and having a slightly larger diameter than a shaft member connecting portion 430 (see FIG. 6) which connects the arm body part 31L and the shaft 400, so as to prevent the left and right arm parts 3R and 3L, which are respectively attached to the right arm supporting member 10R and the left arm supporting member 10L, from falling out from the body part 10.

Further, the panel-side case 510 and the power-side case 520 are produced by injection molding from predetermined resin. In other words, since the attaching position stopping portion 515 is placed at one of the panel-side case 510 and the power-side case 520 and the housing position stopping portion 525 is placed at another, it is possible to have more variance of a position where one of the attaching position stopping portion 515 and the housing position stopping portion 525 is placed than a case of placing both of the attaching position stopping portion 515 and the housing position stopping portion 525 in one of the panel-side case 510 and the power-side case 520. Thereby, it is possible to simplify the structures of the panel-side case 510 and the power-side case 520. That is, by simplifying the injection molding of the panel-side case 510 and the power-side case 520, it is possible to form the panel-side case 510 and the power-side case 520, easily.

The pulse sensor unit 5 is a detecting section for detecting pulse, which is a state of bloodstream, and comprises a clip which can be pinched to an earlobe, a portion of an ear. In the pulse sensor unit 5, a sensor for optically detecting pulse is provided on the pinching surface thereof. Further, the pulse sensor unit 5 is electrically connected to the left side surface of the body part 10 through the cable 50, and is structured to be communicable with a pulse measuring unit 108. Further, while a user is not using the pulse sensor unit 5, the pulse sensor unit 5 is pinched and fixed at a protruding portion 34.

The pulse sensor unit 5 comprises a light emitting device such as a light emitting diode, and a light receiving device such as a photodiode for structuring the sensor for optically detecting pulse. Here, since its mechanism and structure are well-known technologies, its detailed description is omitted.

In order to attach the ear-attaching type device 1, a user holds and widens the right arm part 3R and the left arm part 3L in a direction in which the right driver unit 34R and the left driver unit 34 separate from each other. Then, the user moves the ear-attaching type device 1 so as to go around the head part from the occipital part H side, and the user attaches the ear-attaching type device 1 with himself/herself by inserting the right driver unit 34R into an ear hole of the right ear and the left driver unit 34L into an ear hole of the left ear.

At this time, according to the bias which is transmitted to the right driver unit 34R and the left driver unit 34L through the right arm part 3R and the left arm part 3L respectively, the right driver unit 34R and the left driver unit 34L are biased in a direction of the ear hole (internal direction). Further, as shown in FIG. 1B, the back surface of the body part 10 (a surface at the front side of the body part 10) is contacted with a lower part of the occipital part H, and thereby a posture of the body part 10 is maintained.

[1-2 Effect According to External Structure]

According to such an ear-attaching type device 1, the following effects can be obtained. First, by inserting the right driver unit 34R into an ear hole of the right ear and the left driver unit 34L into an ear hole of the left ear, the right driver unit 34R and the left driver unit 34L are biased in the internal direction of the head part according to the bias transmitted through the right arm part 3R and the left arm part 3L. Thereby, the right driver unit 34R and the left driver unit 34L are surely inserted into ear holes. Accordingly, each driver unit does not easily fall off according to movement of the head part, and thereby it is possible to obtain a sense of stable attachment.

Further, although a line connecting the right driver unit 34R inserted into an ear hole of the right ear and the left driver unit 34L inserted into an ear hole of the left ear can be a pivot shaft according to which the body part 10 is fluctuated in the up-down direction, the misalignment in the shaft direction of the pivot shaft of the ear-attaching type device 1 is suppressed.

Further, since the body part 10 incorporates therein a power unit such as a battery and various control circuits, the body part 10 occupies a major part of the ear-attaching type device 1 in weight, and thereby the body part 10 has certain amount of weight. Therefore, the body part 10 is contacted in the vicinity of the lower part of the occipital part H according to relation between the weight thereof and the pivot shaft. Accordingly, the ear-attaching type device 1 is maintained with a stable posture where the body part 10 is contacted in the vicinity of the lower part of the occipital part H, and thereby the fluctuation in the rotation direction of the pivot shaft is suppressed even if a user is doing the exercise.

Further, since the ear-attaching type device 1 is contacted with the head part with three points, which are left and right ear holes and the occipital part H, it is possible to eliminate surrounding sense over the head part which was caused by the conventional headphone, and thereby it is possible to obtain a comfortable sense of attachment.

Further, the right arm part 3R is biased to the head part from outside of the right ear, and the left arm part 3L is biased to the head part from outside of the left ear. Therefore, since the right arm part 3R does not use a base part of the right ear and the left arm part 3L does not use a base part of the left ear, it is possible to wear a pair of glasses while the ear-attaching type device 1 is being attached.

Further, since the pulse sensor unit 5 connected to the left side surface of the body part 10 is engaged by pinching a left earlobe. Therefore, since the attachment is completed by attaching the ear-attaching type device 1 to the head part of a user, it is possible to reduce the botheration of the cable 50 against exercise that a user is doing.

Further, while the pulse sensor unit 5 is engaged by pinching the left ear (left side), the pulse switch 36R is placed on the right driver unit supporting member 32R (right side). Thereby, it is possible to reduce influence to a pulse detection by the left-side pulse sensor unit 5 from an operation of the pulse switch 36R for listening to a measurement result such as pulse rate or the like, which is done on the right side of the ear-attaching type device 1.

Further, with the flange member 410 and the sliding guide 420 placed at the shaft member 400 having rotation shafts S3 and S5 for the left and right arm parts 3R and 3L respectively, it is possible to rotate the left and right arm parts 3R and 3L with respect to the body part 10, and also it is possible to stop the rotation of the left and right arm parts 3R and 3L by the attaching position stopping portion 515 and the housing position stopping portion 525 which are respectively placed in the panel-side case 510 and the power-side case 520. Therefore, even if the left and right arm parts 3R and 3L are rotated with respect to the body part 10, unreasonable force is not applied to the connecting member 6 placed inside of the left and right arm parts 3R and 3L, whereby it is possible to prevent from breaking the connecting member 6.

Here, it is possible to determine a rotation stopping position of the left and right arm parts 3R and 3L at the time of attaching the ear-attaching type device 1 by the attaching position stopping portion 515, and further it is possible to determine a rotation stopping position of the left and right arm parts 3R and 3L at the time of housing the ear-attaching type device 1 by the housing position stopping portion 525. In other words, by contacting the attaching position rotation stopping surface 422 of the sliding guide 420 with the attaching position stopping portion 515 by rotating the left and right arm parts 3R and 3L, it is possible to stop the left and right arm parts 3R and 3L at the device attaching position of the ear-attaching type device 1. Further, by contacting the housing position rotation stopping surface 423 with the housing position stopping portion 525, it is possible to stop the left and right arm parts 3R and 3L at the device housing position of the ear-attaching type device 1. Therefore, it is possible to attach and house the ear-attaching type device 1 easily.

Further, by the sliding guide 420, it is possible to properly guide the rotation of the left and right arm parts 3R and 3L so as to slide the external surface of the sliding guide 420 against the internal sliding surfaces 516 and 526 each of which respectively corresponds to the panel-side case 510 and the power-side case 520. Therefore, it is possible to rotate the left and right arm parts 3R and 3L more properly.

Further, since the groove 424 with which the ribs 517 and 527 are to be engaged is formed on the internal surface of the sliding guide 420, it is possible to guide the rotation of the left and right arm parts 3R and 3L by the sliding guide 420 more properly, whereby it is possible to suppress tilt, irregular movement and the like of the left and right arm parts 3R and 3L.

Further, since the flange member 410 is formed so as to become thicker as coming close to the rotation shafts S3 and S5 of the left and right arm parts 3R and 3L, it is possible to intensify the strength around the rotation shafts S3 and S5 at the side of the rotation shafts S3 and S5, whereby it is possible to rotate the left and right arm parts 3R and 3L more properly.

Further, since the external surface 411 of the flange member 410 is formed so as to dent toward the side of the rotation shaft S3 and S5, by forming the panel-side internal wall portion 514 of the panel-side case 510 and the power-side internal wall portion 524 of the power-side case 520 so as to follow the shape of the external surface 411, it is possible to more properly secure an implementation range of devices placed inside of the body part. Further, since the external surface 411 of the flange member 410 is formed in a curved shape so as to follow the panel-side internal wall portion 514 and the power-side internal wall portion 524, it is possible to have a large contacting area of the external surface 411 of the flange member 410 with the panel-side internal wall portion 514 and the power-side internal wall portion 524. Therefore, it is possible to guide the rotation of the left and right arm parts 3R and 3L by the flange member 410, whereby it is possible to properly suppress tilt, irregular movement and the like of the left and right arm parts 3R and 3L.

Here, in the embodiment above, described is the case that the rotation of the right arm part 3R and the left arm part 3L is stopped at the device attaching position and the device housing position of the ear-attaching type device 1. However, the present invention is not limited to such a case. A rotation stopping position of the right arm part 3R and the left arm part 3L may be anywhere as long as the rotation of the right arm part 3R and the left arm part 3L can be stopped within one turn.

Further, described is the case that the attaching position stopping portion 515 is placed in the panel-side case 510 as a first body case member, and the housing position stopping portion 525 is placed in the power-side case 520 as a second body case member. However, the present invention is not limited to such a case. For example, the housing position stopping portion 525 may be placed in the power-side case 520, and the attaching position stopping portion 515 may be placed in the power-side case 520.

Here, the rotation stopping mechanism portion may be placed outside of a case member such as the panel-side case 510, the power-side case 520 or the like. For example, rotation of the left and right arm parts 3R and 3L may be stopped with a convex attaching position stopping portion 515 and a convex housing position stopping portion 525 placed outside of a case member, by contacting the left and right arm parts 3R and 3L with these attaching position stopping portion 515 and the housing position stopping portion 525.

Further, in the above-described embodiment, illustrated is the shaft member 400 comprising the flange member 410 and the sliding guide 420, as the right arm part 3R and the left arm part 3L. However, the present invention is not limited to such a case. Whether or not to place the flange member 410 and the sliding guide 420 is suitably changeable according to a shape or the like of objective right arm part 3R and left arm part 3L.

Further, described is the case that the flange member 410 and the sliding guide 420 are united and continuously formed on the shaft member 400. However, the present invention is not limited to such a case. For example, the flange member 410 and the sliding guide 420 may be formed with predetermined distance secured between the two.

In this case, whether a groove 424 with which the ribs 517 and 527 of the body part 10 are to be engaged is provided in the sliding guide 420 is also an optional requirement, and therefore it is suitably changeable according to a shape or the like of the body part 10.

[1-3 Various Switches]

The ear-attaching type device 1 comprises various switches for realizing a function of the inputting unit 60. The body part 10 comprises a mode switch 18a, a start-stop switch 18c for starting or stopping an operation of the stopwatch and an operation of detecting pulse simultaneously, a radio switch 18b for starting reception of the radio, a power switch 18d for turning the power ON/OFF, and a volume switch 18e for changing the volume of sound. Further, the right driver unit supporting member 32L comprises a pulse switch 36R, and the left driver unit supporting member 32R comprises a tuner switch 36L for tuning in the radio.

When the power switch 18d is pushed, the ear-attaching type device 1 is turned on (ON) and enters a pulse measurement capable state. Concretely, first, the mode switch 18a is pushed, and values such as age and the like are inputted. Then, when the start-stop switch 18c is pushed, the ear-attaching type device 1 measures pulse, and starts counting a time period for which the pulse is measured (hereafter, it is suitably referred to as “pulse measurement time”). Further, if the start-stop switch 18c is pushed again during the pulse measurement, the pulse measurement is temporarily suspended and the count of the pulse measurement time is also suspended. Further, by pushing and holding the start-stop switch 18c for a predetermined period (for example, “1 second”), the pulse measurement is stopped and the pulse measurement time is reset. Further, if the pulse switch 36R is pushed during the pulse measurement, the CPU 100 gives a report of a current pulse rate and the like by executing the interruption reporting program 214.

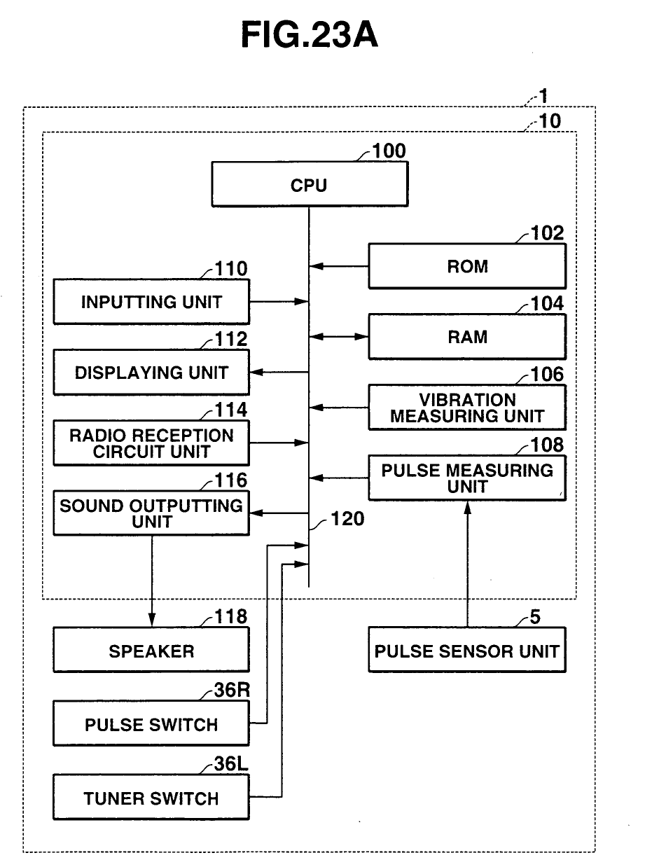

Concretely, description will be made with reference to a state transition diagram of FIG. 22A. FIG. 22A is a view on the screen display 12, showing a state transition of the ear-attaching type device 1. First, when the power is turned ON, the ear-attaching type device 1 enters a state A. The state A indicates that the pulse measurement time is 0 second, and the pulse sensor is OFF. If the start-stop switch 18c is pushed in this state, the ear-attaching type device 1 enters a state B. The state B is a state where the stopwatch is functioning and the pulse measurement time is being counted. At this time, the pulse sensor is turned ON.

If the start-stop switch 18c is pushed in the state B, the ear-attaching type device 1 enters a state C. The state C is a state where the counting of the pulse measurement time is temporarily suspended. At this time, the pulse sensor is turned OFF. If the start-stop switch 18c is pushed at this state, the ear-attaching type device 1 enters the state B again.

Further, if the start-stop switch 18c is pushed and held (holding it down for a long time) in either the state B or the state C, the ear-attaching type device 1 enters the state A, and the pulse measurement time is reset and the pulse sensor is turned OFF.

The radio switch 18b is a switch for operating a radio function. Here, outline of the radio function will be briefly described. First, when a user pushes the radio switch 18b, the ear-attaching type device 1 turns the radio function ON, and outputs broadcasting of a selected reception frequency from the left driver unit 34L and the right driver unit 34R. Further, by pushing the volume switch 18e, volume of sound is adjusted. Further, when the tuner switch 36L is pushed, another reception frequency is selected and the received broadcasting is outputted from the left driver unit 34L and the right driver unit 34R.

[1-4 Internal Structure]

Here, the ear-attaching type device 1 incorporating therein a pulse measurement function will be described. FIG. 13A is a block diagram showing an internal structure of the ear-attaching type device 1. As shown in FIG. 13A, the ear-attaching type device 1 comprises a CPU (Central Processing unit) 100, a ROM (Read Only Memory) 102, a RAM (Random Access Memory) 104, a vibration measuring unit 106, the pulse measuring unit 108, the pulse sensor unit 5, a radio reception circuit unit 114, an inputting unit 110, a displaying unit 112, a sound outputting unit 116, and a signal data line 120.

[1-4-1 ROM]

FIG. 13C shows a structure of data and programs stored in the ROM 102. The ROM 102 is a read only memory which stores an initial program for performing various initial settings, hardware inspection, loading of necessary programs and the like. By executing the initial program at the time of turning on the power of the ear-attaching type device 1, the CPU 100 sets an operation environment of the ear-attaching type device 1.

Further, the ROM 102 stores various programs regarding operations of the ear-attaching type device 1, such as a radio reception process, various setting processes, various communication processes and the like, and further stores an exercise purpose table 202, an advice sound storing area 204, a numeric value sound storing area 206, a device controlling program 208, a first pulse measuring program 210, a sound reporting program 212 and an interruption reporting program 214.

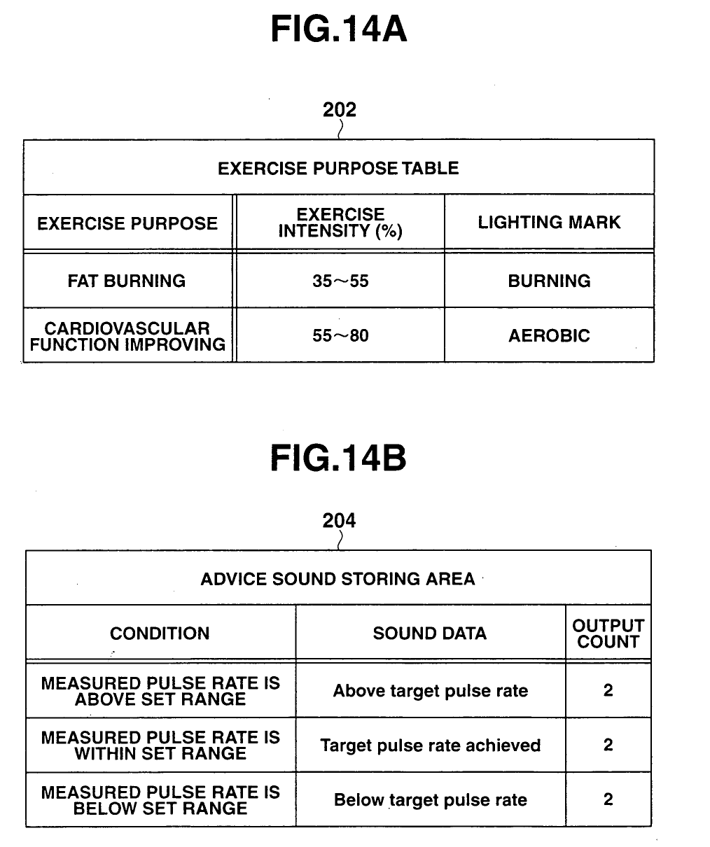

The exercise purpose table 202 is a table for storing parameters regarding “exercise purpose” which indicates an operation mode of the ear-attaching type device 1. As shown in FIG. 14A, the exercise purpose table 202 stores a range of exercise intensity (for example, “35 to 55”) and a lighting mark displayed on the screen display 12 (for example “BURNING”) so as to relate them with an exercise purpose (for example, “FAT BURNING”).

Here, the exercise intensity means a value indicating how much portion (%) a differential between a pulse rate per minute of a user doing the exercise (hereafter, it is suitably referred to as “pulse rate at exercising”) and a pulse rate per minute of a user resting (hereafter, it is suitably referred to as “pulse rate at resting”) occupies out of a differential between the maximum pulse rate and the pulse rate at resting of the user.

The advice sound storing area 204 is an area in which sound data for the CPU 100 to report advice with sound is stored. FIG. 14B is a view describing a data structure of the advice sound storing area 204. In the advice sound storing area 204, sound data for reporting, for example, “Above target pulse rate” is stored. Then, for example, if a condition “MEASURED PULSE RATE IS ABOVE SET RANGE” is satisfied, the sound data “Above target pulse rate” is read out and outputted with sound (reported) as many as “2” times.

The numeric value sound storing area 206 is an area in which sound data corresponding to a numeric value used at the time of reporting pulse rate is stored. For example, sound data “one” corresponding to “1”, sound data “fifty” corresponding to “50” are stored. Then, if “51” is to be reported, the CPU 100 reports pulse rate by outputting “fifty” and “one” continuously with sound.

[1-4-2 RAM]

FIG. 13B is a view showing a data structure accessed in the RAM 104. RAM 104 is a rewritable memory at any time for temporarily storing various programs executed by the CPU 100, data regarding the execution of these programs, and the like. In the present embodiment, in the RAM 104, a pulse cycle accumulation storing area 302, a pulse rate accumulation storing area 304, an individual data 306, and a set range data 308 are secured.

The pulse cycle accumulation storing area 302 is a storing area for storing a time period as much as one pulse takes (hereafter, it is suitably referred to as “pulse time”) regarding the pulse measured by the pulse sensor unit 5 so as to accumulate it. For example, if the pulse time is measured as “400 ms”, the CPU 100 stores “400 ms” in the pulse rate accumulation storing area 304.

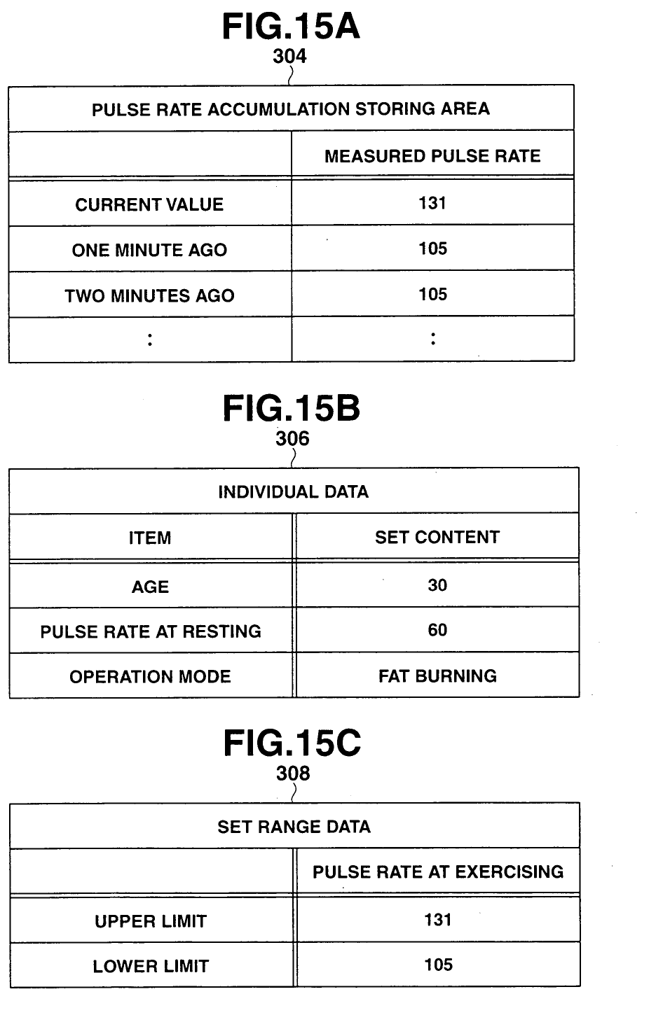

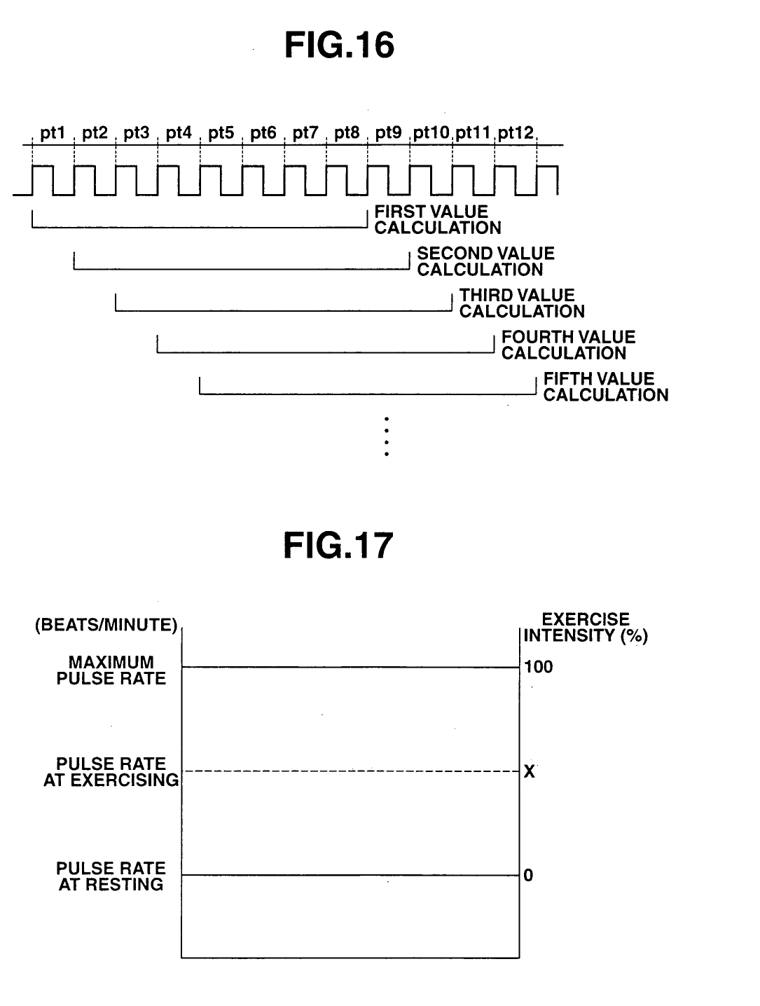

The pulse rate accumulation storing area 304 is an area for storing calculated pulse rate per minute so as to accumulate it. As shown in FIG. 15A, the pulse rate accumulation storing area 304 stores pulse rate per minute which is calculated from measured pulse (hereafter, it is suitably referred to as “measured pulse rate”) at each one minute so as to accumulate it. Here, a method to calculate pulse rate per minute will be described with reference to FIG. 16, equation 1 and equation 2.

FIG. 16 is a view describing a method to calculate pulse rate. In FIG. 16, for descriptive purposes, a pulse of one time (beat) is shown as a waveform of a pulse wave. Further, pulse time for taking one pulse (heartbeat) are respectively shown as “pt1”, “pt2” and the like. Further, in order to calculate pulse rate, it is shown that eight pieces of pulse time from “pt1” to “pt8” are used to calculate a first value (initial value), and eight pieces of pulse time from “pt2” to “pt9” are used to calculate a second value. Here, the illustration is made under the assumption that pulse rate is calculated with a unit of pulse for descriptive purposes. However, in the present embodiment, pulse rate is calculated for each one minute.

The CPU 100 stores pulse time of pulse measured by the pulse sensor unit 5 as needed, in the pulse cycle accumulation storing area 302 so as to accumulate it. Then, among the pieces of pulse time stored in the pulse cycle accumulation storing area 302, the CPU 100 extracts as many as eight pieces (“pt1”, “pt2”, . . . , “pt8”). Then, among the extracted eight pieces of pulse time, the CPU 100 excludes the largest two pieces and the smallest two pieces, calculates the sum of the rest four pieces and divides the sum by four, for calculating a mean of pulse time (hereafter, it is suitably referred to as “mean pulse time”). Then, by dividing sixty by the mean pulse time, pulse rate per the first one minute is calculated. This is the method to calculate for the first time. The equation 1 shows an equation to calculate a pulse rate per the first one minute.
First Value=60/((Sum of pt1 to pt8 excluding largest two and smallest two)/4) Equation 1:

Calculation of a pulse rate of the second time and later will be done in the following way. That is, for example, if a pulse rate of the second time is calculated based on as many as eight pieces of pulse rate from “pt2” to “pt9”, mean pulse time of four pieces of pulse time, which are the eight pieces of pulse time excluding the largest two pieces and the smallest two pieces. Then, the CPU 100 calculates a pulse rate of the second time by calculating the sum of the calculated second mean pulse time and a value which is the mean pulse time calculated formerly (the first time) multiplied by three as weighing, dividing the sum by four and dividing 60 by the divided value. The equation 2 shows an equation to calculate a pulse rate of the second time and later.
Second Value and later=60/(((Sum of pt1 to pt8 excluding largest two and smallest two)+(formerly calculated three values))/4) Equation 2:

Similarly, as well as the third time and later, by doing the calculation based on the equation 2, it is possible to calculate a pulse rate. Here, in the present embodiment, the description is made by illustrating the case that a timing of calculating a pulse rate is each one minute. However, the present invention is not limited to such a case. For example, the calculation may be done for each five minutes, or may be done always. Here, since, in general, a pulse rate does not make a sudden drastic change, if the calculation is done for each one minute, it is possible to reduce processing loads on the CPU 100 compared to the case of doing the calculation always, whereby it is possible to make a battery life longer.

The individual data 306 stores information of a user. As shown in FIG. 15B, the individual data includes age (for example, “30”), pulse rate at resting (for example, “60”), and an operation mode (for example, “FAT BURNING”). These information are inputted by the user.

The set range data 308 is data for storing a range of a pulse rate at exercising with respect to a range of exercise intensity which is appropriate for an exercise purpose as a set range. As shown in FIG. 15C, the set range data 308 stores an upper limit of a pulse rate at exercising (for example, “131”), and a lower limit (for example, “105).

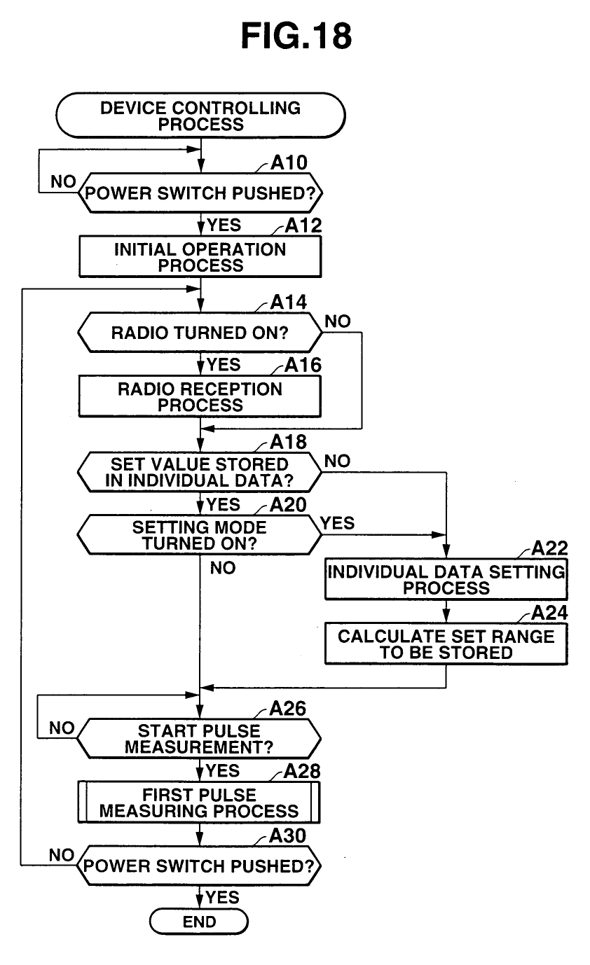

Here, a method to calculate a set range will be described concretely with reference to FIG. 17, equation 3 and equation 4. As shown in FIG. 17, a maximum pulse rate is set as exercise intensity of 100%, and a pulse rate at resting is set as exercise intensity of 0%. Here, the maximum pulse rate is an upper limit of a pulse rate when a user exercises, and it is possible to calculated it by “220−AGE”. Further, the pulse rate at resting is a value of a pulse rate measured when a user is in a resting state, and corresponds to “PULSE RATE AT RESTING” stored in the individual data 306.

The exercise intensity is, as described, a value indicating how much portion (%) a differential between a pulse rate per minute of a user doing the exercise (pulse rate at exercising) and a pulse rate per minute of a user resting (pulse rate at resting) occupies among a differential between the maximum pulse rate and the pulse rate the resting of the user. Concretely, exercise intensity is a value (%) obtained by dividing a value obtained by subtracting the pulse rate at resting from the pulse rate at exercising by a value obtained by subtracting the pulse rate at resting from the maximum pulse rate, and by multiplying the divided value by 100. The equation 3 is an equation to calculate exercise intensity.
Exercise intensity=((pulse rate at exercising−pulse rate at resting)/(maximum pulse rate−pulse rate at resting)×100 Equation 3:

Further, the pulse rate at exercising is calculated by the followings. First, a value obtained by subtracting the pulse rate at resting from the maximum pulse rate is multiplied by the exercise intensity. Then, the multiplied value is divided by 100 and the pulse rate at resting is added. The equation 4 shows an equation to calculate the pulse rate at exercising.
Pulse rate at exercising=((maximum pulse rate−pulse rate at resting)×exercise intensity/100)+pulse rate at resting Equation 4:

For example, as shown in FIG. 15B, if the individual data 306 stores age of “30” years old, a pulse rate at resting of “60” and an operation mode of “FAT BURNING”, a set range is calculated in the following way. First, the maximum pulse rate is “220−30=190”. Then, since the operation mode is “FAT BURNING”, the range of exercise intensity is “35 to 55” according to the exercise purpose table 202. At first, when a pulse rate at exercising with the exercise intensity “35”% is calculated, the result is “((220−30)−60)×35/100+60=105”. Further, if a pulse rate at exercising with the exercise intensity “55”% is calculated, the result is “((220−30)−60)×55/100+60=131”. Therefore, the set range of the pulse rate at exercising is stored in the set range data 308 with a lower limit set as 105 and an upper limit set as 131.

[1-4-3 CPU]

The CPU 100 is a central processing unit which performs giving an instruction to each function unit and transmitting of data by executing processes based on a predetermined program according to an input instruction. Concretely, CPU 100 reads out a program stored in the ROM 102 according to an operation signal inputted from the inputting unit 110, and executes a process according to the program. Then, the CPU 100 outputs a display control signal to the displaying unit 112 suitably for displaying a processing result.

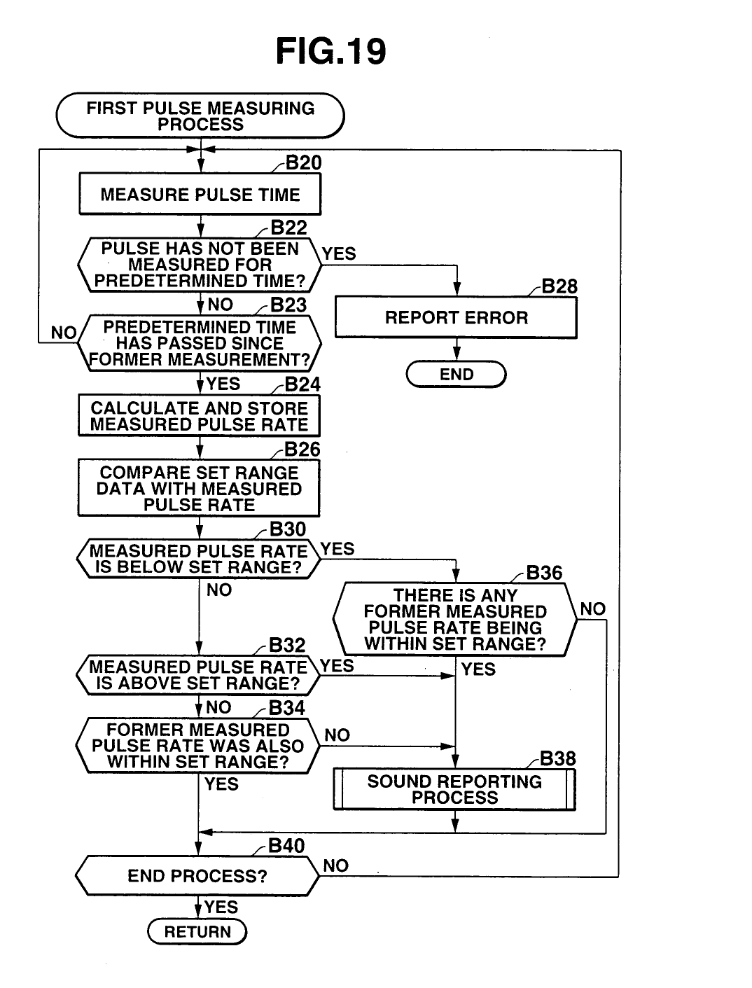

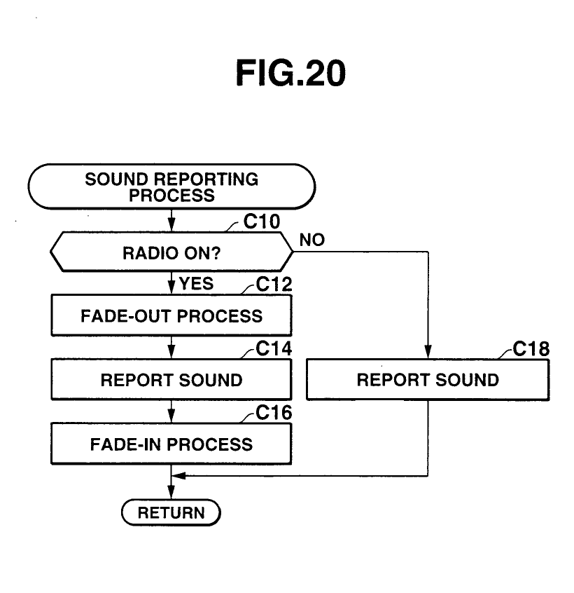

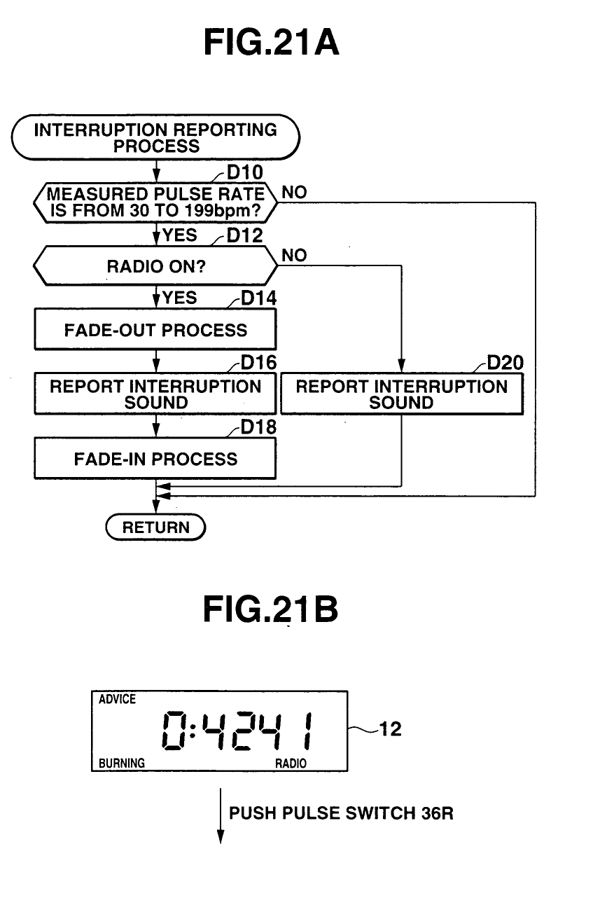

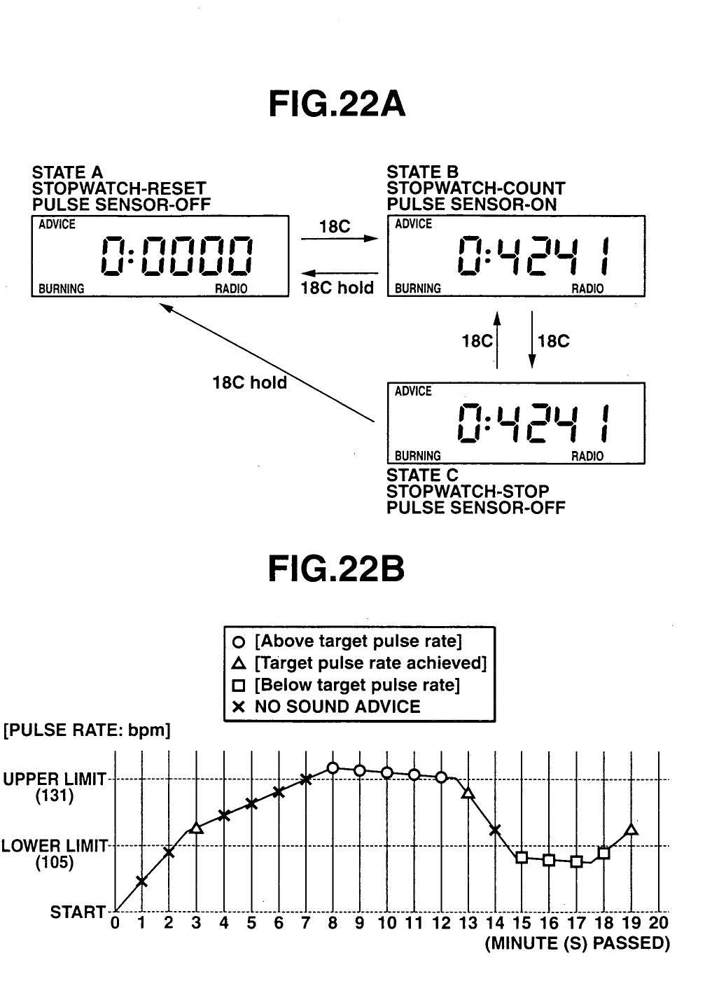

Further, in the present embodiment, the CPU 100 executes a device controlling process (see FIG. 18) according to a device controlling program 208 of the ROM 102, and further executes a first pulse measuring process (see FIG. 19) according to a first pulse measuring program 210 and a sound reporting process (see FIG. 20) according to a sound reporting program 212, as a subroutine. Further, when the pulse switch 36R is pushed, the CPU 100 executes an interruption reporting process (see FIG. 21A) according to an interruption reporting program 214 as an interruption process.

Concretely, in the device controlling process, when the power switch 18d is pushed, the CPU 100 executes the initial operation process to operate the ear-attaching type device 1. Then, when the radio switch 18b is pushed, the CPU 100 receives the radio. Then, if the individual data 306 is not stored or if the setting mode is turned ON, the CPU 100 lets a user to input individual data 306. Then, based on the inputted individual data 306 of the user, the CPU 100 calculates a set range to be stored as the set range data 308. Then, if the start-stop switch 18c is pushed, the CPU 100 executes the first pulse measuring process. Then, if the power switch 18d is pushed, the CPU 100 finishes the device controlling process.

Further, in the first pulse measuring process, the CPU 100 measures pulse time corresponding to one time of pulse and stores it in the pulse cycle accumulation storing area 302 so as to accumulate it, at each time that the pulse sensor unit 5 measures (detects) pulse. Further, for each time that a predetermined time period has passed since the former measurement (concretely, for each one minute), the CPU 100 calculates a measured pulse rate based on the pulse time stored in the pulse cycle accumulation storing area 302. Then, by judging whether it is within the range of the pulse rate at exercising stored in the pulse cycle accumulation storing area 302 or not, the CPU 100 executes the sound reporting process which performs the sound reporting according to a judgment result.

Further, in the sound reporting process, if the radio is ON, the CPU 100 gradually decreases volume of the sound output of the radio to perform the sound reporting. Then, when the sound reporting is completed, the CPU 100 gradually increases volume of the sound output of the radio to do the outputting with the same volume as before the sound reporting.

Further, when the pulse switch 36R is pushed, the CPU 100 executes the interruption reporting process. In the interruption reporting process, the CPU 100 judges whether the measured pulse rate is included within the pulse rate reporting range, which is from 30 to 199. Then, if the measured pulse rate is from 30 to 199, the CPU 100 further judges whether the radio is ON or not. If the radio is ON, the CPU 100 gradually decreases volume of the sound output of the radio to perform the sound reporting. Then, when the sound reporting is completed, the CPU 100 gradually increases volume of the sound output of the radio to perform the sound output with the same volume as before the sound reporting.

[1-4-4 Pulse sensor unit]

The pulse sensor unit 5 is a device for detecting and measuring pulse (heartbeat) by measuring a bloodstream state of a user. As shown in FIG. 2A, when a clip is pinched to a ear of the user, the pulse sensor placed in the clip detects pulse by contacting with the ear of the user. Here, the pulse sensor comprises a light emitting device such as a light emitting diode, and a light receiving device such as a phototransistor and the like. Further, the pulse sensor emits light toward inside (ear side) from the light emitting device, and with the emitted light reflected by the contacting ear and the reflected light received by the light receiving device, the pulse sensor detects density change of hemoglobin in blood transmitted to a blood vessel of the ear, according to a beat of a heart. The CPU 100 measures (detects) pulse time based on a detection signal of the pulse sensor unit 5.

Here, light emitted from the light emitting device toward inside (ear side) may be transmitted through the contacting ear and the transmitted light may be received by the light receiving device.

[1-4-5 Radio reception unit]

The radio reception circuit unit 114 is a circuit which outputs sound data of broadcasting contents by receiving and demodulating radio wave transmitted from a broadcasting station. The CPU 100 receives radio wave of a broadcasting station (frequency) set by a user, and demodulates it as a sound signal. Here, since its detailed technology content is well known, the description thereof is omitted.

[1-4-6 Inputting-Outputting Unit]

The inputting unit 110 is an inputting device comprising switches which are necessary for selecting a function, and outputs a signal of a pushed switch to the CPU 100. By the switch input on the inputting unit 110, the inputting section of a control command for instructing a process execution or the like is realized. Here, the inputting unit 110 is equivalent to various switches such as the power switch 18d and the like shown in FIG. 2A.

The displaying unit 112 displays various screens based on a display signal outputted from the CPU 100, and comprises an LCD (Liquid Crystal Display) or the like. Here, the displaying unit 112 is equivalent to the screen display 12 in FIG. 2A.

The sound outputting unit 116 outputs sound according to a sound signal outputted from the CPU 100, and comprises a speaker, an earphone and the like. Here, the sound outputting unit 116 is equivalent to the left driver unit 34L and the right driver unit 34R in FIG. 2A.

The signal data line 120 is a line for transmitting an electrical signal such as various data signals, control signals and the like, and is a signal line for connecting each of the CPU 100, the ROM 102, the RAM 104, the pulse sensor unit 5, the inputting unit 110, the displaying unit 112 and the sound outputting unit 116.

[1-5 Operation]

[1-5-1 Device Controlling Process]

First, the device controlling process will be described. FIG. 18 is a flowchart illustrating an operation of the ear-attaching type device 1 according to the device controlling process. The device controlling process is a process which is realized with the CPU 100 executing the device controlling program 208 stored in the ROM 102.

First, when the power switch 18d of the ear-attaching type device 1 is pushed (Step A10; Yes), the CPU 100 executes the initial operation process such as initializing various variables (Step A12).

Next, when the radio switch 18b is pushed (Step A14; Yes), the CPU 100 receives and demodulates radio broadcasting tuned in by a user through the radio reception circuit unit 114, and outputs it from the sound outputting unit 116 (Step A16).

Next, the CPU 100 judges whether a set value is stored in the individual data 306 or not (Step A18). Here, if a set value is not stored in the individual data 306 (Step A18; No), the CPU 100 lets a user input age, a pulse rate at resting and an operation mode to be stored in the individual data 306 (Step A22). Then, according to a value stored in the individual data 306, the CPU 100 calculates a set range of pulse with respect to exercise intensity, and stored it in the set range data 308 (Step A24).

If a set value is stored in the individual data 306 (Step A18; Yes), the CPU 100 judges whether a setting mode is turned ON or not with a user pushing the mode switch 18a (Step A20). Then, if a user turns the set mode ON (Step A20; Yes), the CPU 100 stores the set value in the individual data 306 instructed and inputted by the user (Step A22, A24).

Next, when the start-stop switch 18c is pushed (Step A26; Yes), the CPU 100 starts the first pulse measuring process (Step A28). Then, when the power switch 18d is pushed, the CPU 100 stops the operation of the ear-attaching type device 1 (Step A30).

[1-5-2 First Pulse Measuring Process]

Next, the first pulse measuring process will be described. FIG. 19 is a flowchart illustrating an operation of the ear-attaching type device 1 according to the first pulse measuring process. The first pulse measuring process is a process realized with the CPU 100 executing the first pulse measuring program 210 stored in the ROM 102, and it is executed in Step A28 of the device controlling process.

First, the CPU 100 measures time per one time of pulse (pulse time) measured (detected) by the pulse sensor unit 5 (Step B20). Here, if pulse time is not measured for a predetermined time (Step B22; Yes), the CPU 100 performs an error reporting and ends the process (Step B28). For example, when pulse time is not measured for two minutes, a user is notified that pulse time is not measured (pulse is not detected), by outputting reporting sound “error” from the sound outputting unit 116.

Here, for example, the reporting sound “error” may be outputted from the sound outputting unit 116 if the phenomenon that pulse time is not measured for two minutes happens two times.

Then, when pulse time of a user is measured (Step B22; No), the CPU 100 judges whether a predetermined time has passed since the former measurement or not is judged (Step B23). Here, if the predetermined time has not passed (Step B23; No), the CPU 100 executes processes from Step B20 again. On the contrary, if the predetermined time has passed (Step B23; Yes), the CPU 100 calculates a pulse rate per minute and stores it in the pulse rate accumulation storing area 304 so as to accumulate it (Step B24). Then, the CPU 100 compares the measured pulse rate with the set range of the pulse rate stored in the set range data 308 (Step B26). Then, if the measured pulse rate is below the lower limit stored in the set range data 308 (Step B30; Yes), the CPU 100 judges whether there has been at least one measured pulse rate being within the set range or not (Step B36). Concretely, the CPU 100 judges whether a value stored in the pulse rate accumulation storing area 304 is included between a lower limit and an upper limit of the set range data 308. Then, if at least one piece of data exists within the set range among pulse rates stored in the pulse rate accumulation storing area 304 (Step B36; Yes), the CPU 100 executes the sound reporting process (Step B38).

Then, when a user selects to end the process (Step B40), the CPU 100 ends the first pulse measuring process, and get the control back to the device controlling process.

[1-5-3 Sound Reporting Process]

Next, the sound reporting process will be described. FIG. 20 is a flowchart illustrating an operation of the ear-attaching type device 1 according to the sound reporting process. The sound reporting process is a process realized with the CPU 100 executing the sound reporting program 212 stored in the ROM 102, and it is executed in Step B38 of the first pulse measuring process.

First, the CPU 100 judges whether the radio is ON or not (Step C10). If the radio is ON (Step C10; Yes), the CPU 100 executes a fade-out process (Step C12). Then, the CPU 100 performs the sound reporting (Step C14), and after the sound reporting is completed, the CPU 100 executes a fade-in process to output sound of the radio at the same sound output level as before the sound reporting (Step C16). On the contrary, if the radio is OFF (Step C10; No), the CPU 100 performs the sound reporting (Step C18).

Here, the fade-out process means gradually decreasing a sound output level (volume) from a current sound output level. Further, the fade-in process means gradually increasing a sound output level. Concretely, as a sound output level, ten levels are defined including a level where no sound is outputted is defined as “0”, and a level where output sound is maximum is defined as “9”. Then, if the fade-out process is executed while a current sound output level is “5”, the CPU 100 gradually decreases from “5” to “0”. Further, if the fade-in process is executed thereafter, the CPU 100 gradually increases from “0” to “5”.

The first pulse measuring process will be concretely described with reference to FIG. 22B. FIG. 22B is a graph showing a transition of a pulse rate, with horizontal axis showing time (unit: minute) and vertical axis showing measured pulse rate (unit: bpm: beats per minute). Further, dotted lines are drawn at locations of an upper limit “131” and a lower limit “105” of the set range stored in the set range data 308.

First, measured pulse rates at “1 MINITE PASSED” and “2 MINUTES PASSED” are below the set range (below lower limit), and there is no former pulse rate being within the set range (Step B30; Yes→Step B36; No). Therefore, the CPU 100 does not perform the sound reporting. Next, a measured pulse rate at “3 MINUTES PASSED” is within the set range, and the former pulse rate at “2 MINUTES PASSED” is not within the set range (Ste B30; No→Step B34; No). Therefore, the CPU 100 performs the sound reporting. Here, with reference to FIG. 14B, since the condition “MEASURED PULSE RATE IS WITHIN SET RANGE” is satisfied, the CPU 100 reads out the sound data “Target pulse rate achieved” and reports it from the sound outputting unit 116. Next, a pulse rate at “4 MINUTES PASSED” is within the set range, and the former pulse rate at “3 MINUTES PASSED” is also within the set range (Step B30; No→Step B32; No→Step B34; Yes). Therefore, the CPU 100 does not perform the sound reporting.

Further, a measured pulse rate at “8 MINUTES PASSED” is above the upper limit of the set range (Step B30; No→Step B32; Yes). Therefore, the CPU 100 performs the sound reporting. Here, with reference to FIG. 14B, since the condition “MEASURED PULSE RATE IS ABOVE SET RANGE” is satisfied, the CPU 100 reads out the sound data “Above target pulse rate” and outputs it from the sound outputting unit 116.

Further, a measured pulse rate at “15 MINUTES PASSED” is below the lower limit of the set range. Further, there is a former pulse rate being within the set range such as one at “14 MINUTES PASSED”, the CPU 100 performs the sound reporting (Step B30; Yes→Step→B36; Yes). Here, with reference to FIG. 14B, since the condition “MEASURED PULSE RATE IS BELOW SET RANGE” is satisfied, the CPU 100 reads out the sound data “Below target pulse rate” and outputs it from the sound outputting unit 116.

In this way, in the graph, the mark ‘X’ means a measuring time at which no sound advice is performed, the mark ‘◯’ indicates “MEASURED PULSE RATE IS ABOVE SET RANGE” and means a measuring time at which the sound advice “Above target pulse rate” is outputted, the mark ‘Δ’ indicates “MEASURED PULSE RATE IS WITHIN SET RANGE” and means a measuring time at which the sound advice “Target pulse rate achieved” is outputted, and the mark ‘□’ indicates “MEASURED PULSE RATE IS BELOW SET RANGE” and means a measuring time at which the sound advice “Below target pulse rate” is outputted.

[1-5-4 Interruption Reporting Process]

Next, the interruption reporting process will be described. FIG. 21A is a flowchart illustrating an operation of the ear-attaching type device 1 according to the interruption reporting process. The interruption reporting process is a process realized with the CPU 100 executing the interruption reporting program 214 stored in the ROM 102, and is a process executed as an interruption process by pushing the pulse switch 36R.

First, if a measured pulse rate is within a range from 30 to 199 bpm (Step D10; Yes), the CPU judges whether the radio is ON or not (Step D12). If the radio is ON (Step D12; Yes), the CPU 100 executes the fade-out process (Step D14). Then, the CPU 100 performs a interruption sound reporting (Step D16), and after the interruption sound reporting is completed, the CPU 100 executes the fade-in process to output radio sound at the same sound output level as before the interruption sound reporting (Step D18). On the contrary, if the radio is OFF (Step D12; No), the CPU 100 performs the interruption sound reporting (step D20).

Here, the interruption sound reporting means outputting sound data read out from the advice sound storing area 204 and executing a process to report a measured pulse rate with the sound.

FIG. 21B is a view showing one example of the screen display 12, indicating that pulse is being measured. In this state, if the pulse switch 36R is pushed, the CPU 100 executes the interruption reporting process to report interruption sound. As the interruption sound, for example, “145 (one forty five). Above target pulse rate” is reported. That is, when a current measured pulse rate is “145” bpm, sound data for reporting “145” is read out from the numeric value sound storing area 206 to output “one forty five”, and then sound data corresponding to the current condition is read our from the advice sound storing area 204 to output “Above target pulse rate”.

In this way, according to the first embodiment, the ear-attaching type device 1 is capable of measuring pulse alone. Further, by outputting advice sound “Above target pulse rate” from the ear-attaching type device 1, a user can recognize that the pulse rate is below the set range. Further, by outputting advice sound “Target pulse rate achieved”, a user can recognize that the pulse rate has entered the set range. Therefore, it is possible to adjust exercise amount according to reported sound. Further, even during listening to the radio, it is easy to hear advice sound and the like since sound volume of the radio is automatically adjusted while advice sound or a pulse rate is being outputted.

### SECOND EMBODIMENT

Next, a second embodiment to which the present invention is applied will be described. The present embodiment is to change an interval of pitch sound which is outputted according to a pulse rate at exercising (heartbeat at exercising) according to whether it is within, above or below a set range, in order to achieve appropriate exercise.

[2-1 Structure]

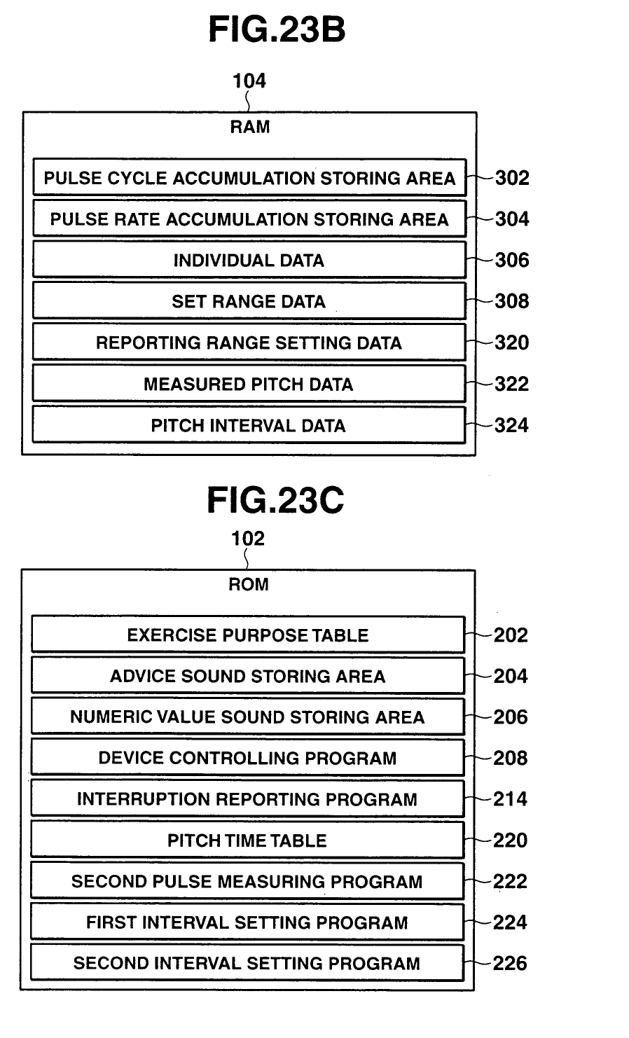

FIG. 23A is a block diagram showing an ear-attaching type device 1 incorporating therein a pulse measuring function. As shown in FIG. 23A, the ear-attaching type device 1 comprises a CPU 100, a ROM 102, a RAM 104, a pulse measuring unit 108, a pulse sensor unit 5, a vibration measuring unit 106, a radio reception circuit unit 114, an inputting unit 110, a displaying unit 112, a sound outputting unit 116, and a signal data line 120. Hereinafter, the same numerals are added to the same components as the first embodiment and the description thereof is omitted. Further, in each flowchart, the same numerals are added to steps having the same processing contents as the flowcharts in the first embodiments, and description thereof will be made in regard to different parts.

Further, FIG. 23B shows a data structure accessed in the RAM 104 in the second embodiment. Further, FIG. 23C shows a structure of data and programs stored in the ROM 102 in the second embodiment.

First, a structure of the ROM 102 will be described. As shown in FIG. 23C, the ROM 102 comprises an exercise purpose table 202, an advice sound storing area 204, a numeric value sound storing area 206, a device controlling program 208, an interruption reporting program 214, a pitch time table 220, a second pulse measuring program 222, a first interval setting program 224 and a second interval setting program 226.

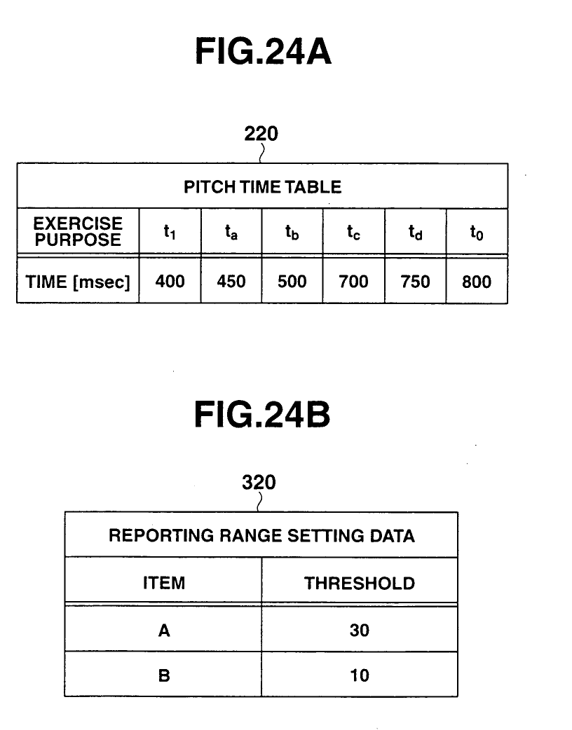

The pitch time table 220 is a table in which time of an interval according to which pitch sound is outputted (hereafter, it is suitably referred to as “pitch sound interval sound”). FIG. 24A shows a data structure of the pitch time table. For example, 400 msec is stored as pitch sound interval time corresponding to time t1, and 500 msec is stored as pitch sound interval time corresponding to time tb.

The second pulse measuring program 222 is a program for realizing the second pulse measuring process in the present embodiment, and the second pulse measuring process is realized with the CPU 100 executing the second pulse measuring program 222. First, each time that the pulse sensor unit 5 measures (detects) pulse, the CPU 100 measures pulse time with respect to the one time pulse and has it stored in the pulse cycle accumulation storing area 302 so as to accumulate it. Further, each time that a predetermined time has passed since the former measurement (concretely, every one minute), the CPU 100 calculates a measured pulse rate based on the pulse time stored in the pulse cycle accumulation storing area 302. Then, the CPU 100 compares a range of a pulse rate stored in the set range data 308 with the calculated measured pulse rate, and if the measured pulse rate is below the set range, the CPU 100 executes the first interval setting process, and if the measured pulse rate is above the set range, the CPU 100 executes the second interval setting process, for outputting pitch sound based on pitch interval data. Further, if a measured pulse rate is within the set range for a predetermined time continuously, the CPU 100 stops the output of pitch sound.

The first interval setting program 224 is a program to realize the first interval setting process in the present embodiment, and the first interval setting process is realized with the CPU 100 executing the first interval setting program 224. The CPU 100 calculates a differential from a lower limit of the set range data 308 to the measured pulse rate. Then, if the calculated differential is not more than a threshold of an item B stored in the reporting range setting data 320, pitch interval time is set to tb, if the calculated differential is more than the threshold of the item B and not more than a threshold of an item A, pitch interval time is set to ta, and if the calculated differential is not less than the threshold of the item A, pitch interval time is set to t1.

The second interval setting program 226 is a program to realize the second interval setting process in the present embodiment, and the second interval setting process is realized with the CPU 100 executing the second interval setting program 226. The CPU 100 calculates a differential from an upper limit of the set range data 308 to the measured pulse rate. Then, if the calculated differential is not more than the threshold of the item B stored in the reporting range setting data 320, pitch interval time is set to td, if the calculated differential is more than the threshold of the item B and not more than the threshold of the item A, pitch interval time is set to td, and if the calculated differential is more than the threshold of the item A, pitch interval time is set to t0.

Continuously, a structure of the RAM 104 will be described. As shown in FIG. 23B, the RAM 104 comprises a pulse cycle accumulation storing area 302, a pulse rate accumulation storing area 304, an individual data 306, a set range data 308, a reporting range setting data 320, a measured pitch data 322 and a pitch interval data 324.

The reporting set range data 320 is an area storing a threshold for calculating how far away it is from either the upper limit or the lower limit of the set range stored in the set range data 308. As shown in FIG. 24B, in the reporting range setting data 308, a threshold of an item A (for example, “30”) and a threshold of item B (for example, “10”) are stored.

The measured pitch data 322 is data storing measured pitch sound interval time. The CPU 100 stores pitch sound interval time at the time of exercising, calculated in Step E12 of the second pulse measuring process (which will be described later), as the measured pitch data 322.

The pitch interval data 324 is data storing pitch sound interval time. The CPU 100 outputs pitch sound from the sound outputting unit 116 based on the pitch interval data 324.

The vibration measuring unit 106 is a function unit for detecting vibration when a user walks or jogs, and it comprises an acceleration sensor and the like. The acceleration sensor may be one according to any one of well known technologies such as strain gage, piezoelectric element and the like.

[2-2 Operation]

[2-2-1 Second Pulse Measuring Process]

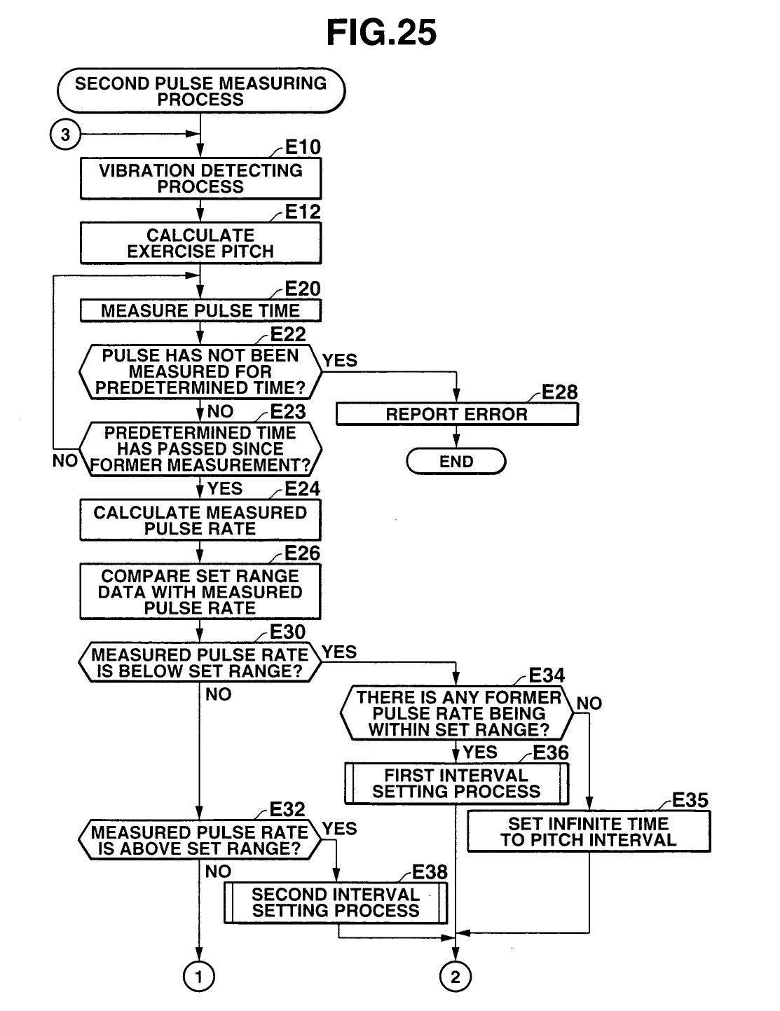

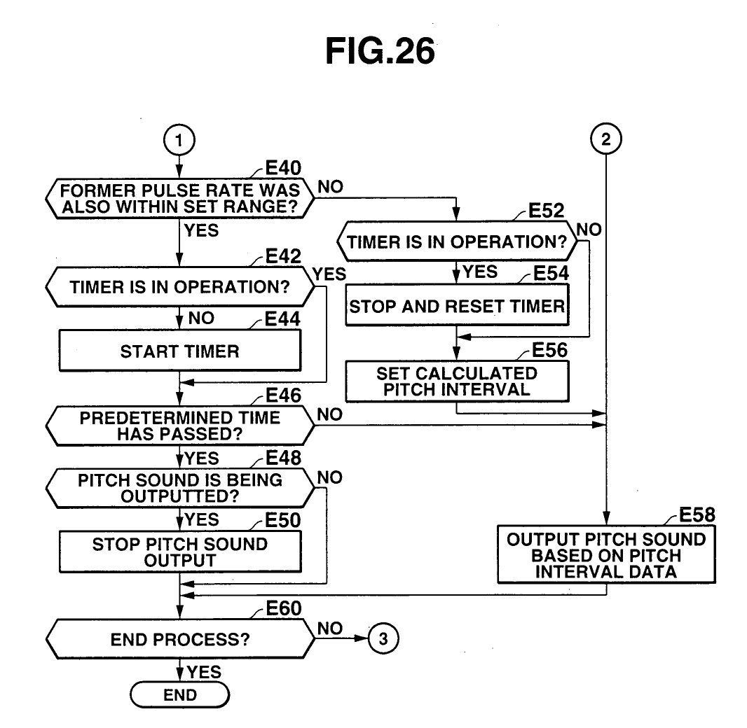

Next, an operation of the ear-attaching type device 1 in the second embodiment will be described with reference to figures. FIGS. 25 and 26 are a flowchart illustrating an operation of the ear-attaching type device 1 according to the second pulse measuring process. The second pulse measuring process is a process realized with the CPU 100 executing the second pulse measuring program 222 stored in the ROM 102, and is executed in Step A28 of FIG. 18 as a subroutine of the device controlling program 208.

First, by detecting vibration of a user with the vibration detecting unit 106 (Step E10), the CPU 100 calculates pitch of current exercise (for example, walking or jogging) and stores it in the measured pitch data 322 (Step E12). Here, as a method to calculate exercise pitch, for example, any one of well-known technologies such as, detecting the count of vibration within five seconds and calculates time per one count of vibration, and the like may be used.

Here, processes from Step E20 to Step E26 are the same as the processes from Step B20 to Step B26 in the first pulse measuring process in the first embodiment. As a brief description thereof, the CPU 100 measures pulse time which is measured (detected) by the pulse sensor unit 5. Then, if a predetermined time has passed since the former measurement, the CPU 100 calculates a pulse rate per minute and stores it in the pulse rate accumulation storing area 304 so as to accumulate it.

Continuously, the CPU 100 compares the measured pulse rate with the set range data 308. Hereinafter, description will be made regarding three cases: (1) when the measured pulse rate is below the lower limit of the set range data 308; (2) when the measured pulse rate is above the upper limit; and (3) when the measured pulse rate is included in the set range.

(1) When the Measured Pulse Rate is Below the Set Range:

When the measured pulse rate is below the lower limit of the set range data 308 (Step E30; Yes), the CPU 100 judges whether there is any former measured pulse rate being within the set range. Concretely, among the pulse rates accumulated and stored in the pulse rate accumulation storing area 304, the CPU 100 judges whether there is any pulse rate being not less than the lower limit and not more than the upper limit of the set range data 308 (Step E34).

Here, if the CPU 100 judges that there is a pulse rate accumulated and stored in the pulse rate accumulation storing area 304, the pulse rate being not less than the lower limit and not more than the upper limit of the set range data 308 (Step E34; Yes), the CPU 100 executes the first interval setting process to set the pitch interval data 324 (Step E36) Then, the CPU 100 outputs pitch sound based on pitch sound interval time set in the pitch interval data 324 (Step E58).

On the contrary, if the CPU 100 judges that there is no pulse rate accumulated and stored in the pulse rate accumulation storing area 304, the pulse rate being not less than the lower limit and not more than the upper limit of the set range data 308 (Step E34; No), the CPU 100 sets infinite time to the pitch interval data 324 (Step E35). Then, the CPU 100 does not output pitch sound due to the fact that infinite time is set to the pitch interval data 324 (Step E58).

(2) When the Measured Pulse Rate is Above the Set Range:

Further, if the measured pulse rate is not below the lower limit of the set range data 308 (Step E30; No) but is above the upper limit of the set range data 308 (Step E32; Yes), the CPU 100 executes the second interval setting process to set the pitch interval data 324 (Step E38). Then, the CPU 100 outputs pitch sound based on pitch sound interval time set in the pitch interval data 324 (Step E58).

(3) When the Measured Pulse Rate is Within the Set Range:

Further, if the measured pulse rate is not less than the lower limit and not more than the upper limit of the set range data 308 (Step E30; No→Step B32; No), the CPU 100 judges whether the former measured pulse rate is not less than the lower limit and not more than the upper limit of the set range data 308 (Step E40).

Here, if the former pulse rate is not less than the lower limit and not more than the upper limit of the set range data 308 (Step E40; Yes), the CPU 100 judges whether timer is in operation (Step E42). Then, if the timer is not in operation (Step E42; No), the CPU 100 starts the timer (Step E44). Then, when the timer counts predetermined time (for example, “2 minutes”) (Step E46; Yes), if pitch sound is being outputted (Step E48; Yes), the CPU 100 stops the output of pitch sound (Step E50). Further, if the timer has not counted the predetermined time (Step E46; No), the CPU 100 outputs pitch sound based on pitch sound interval time set in the pitch interval data 324 (Step E58).

On the contrary, if the former pulse rate is not within a range being not less than the lower limit and not more than the upper limit of the set range data 308 (Step E40; No), when the time is in operation (Step E52), the CPU 100 stops the timer and resets a value of the timer (Step E54). Then, the CPU 100 sets pitch sound interval time stored in the measured pitch data 322 to the pitch interval data 324 (Step E56) and outputs pitch sound (Step E58).

Then, if ending of the process is selected, the CPU 100 ends the second pulse measuring process (Step E60; Yes). If ending of the process is not selected, the process is repeated from Step E10 (Step E60; No).

[2-2-2 First Interval Setting Process]

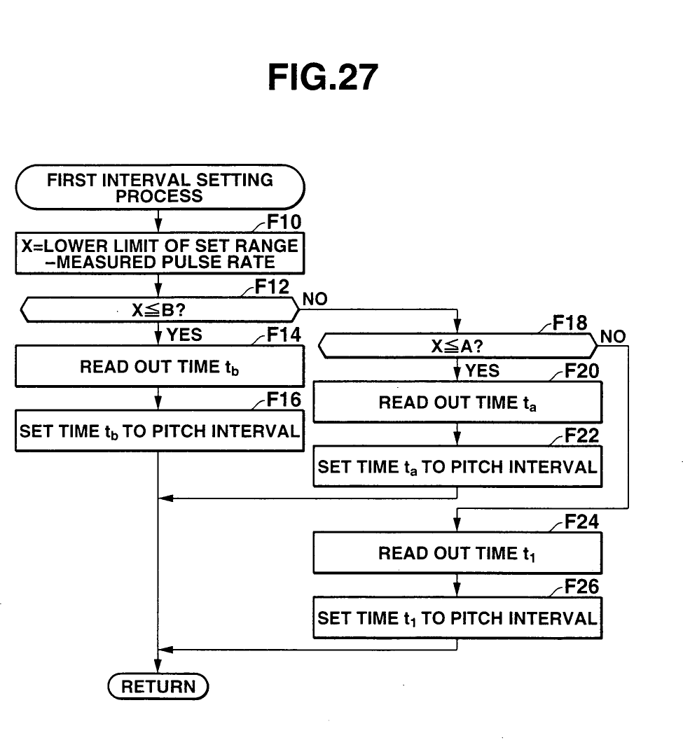

Next, the first interval setting process will be described. FIG. 27 is a flowchart illustrating an operation of the ear-attaching type device 1 according to the first interval setting process. The first interval setting process is a process realized with the CPU 100 executing the first interval setting program 224 stored in the ROM 102, and it is executed in Step E36 of the second pulse measuring process.

First, the CPU 100 assigns a value obtained by subtracting the measured pulse rate from the lower limit of the set range as a variable X (Step F10). Next, if X is not more than a threshold of an item B stored in the reporting range setting data 320 (Step F12; Yes), the CPU 100 reads out the time tb from the pitch time table 220 (Step F14). Then, the CPU 100 sets the time tb to the pitch interval data 324 (Step F16).

On the contrary, if X is more than the threshold of the item B stored in the reporting range setting data 320 (Step F12; No), the CPU 100 judges whether X is not more than a threshold of an item A stored in the reporting range setting data 320 (Step F18). If X is not more than the threshold of the item A stored in the reporting range setting data 320 (Step F18; Yes), the CPU 100 reads out the time ta from the pitch time table 220 (Step F20). Then, the CPU 100 sets the time ta to the pitch interval data 324 (Step F22).

Further, if X is more than the threshold of the item A (Step F18; No), the CPU 100 reads out the time t1 from the pitch time table 220 (Step F20). Then, the CPU 100 sets the time t1 to the pitch interval data 324 (Step F22).

[2-2-3 Second Interval Setting Process]

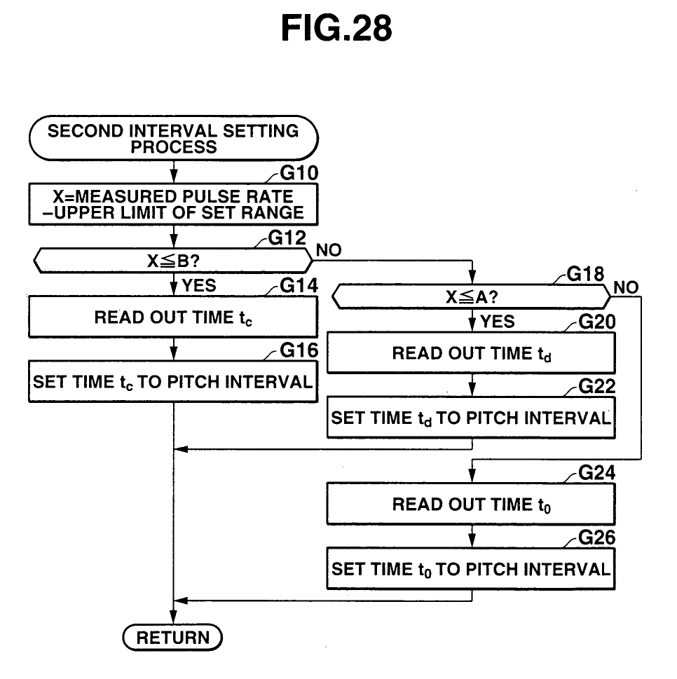

Next, the second interval setting process will be described. FIG. 28 is a flowchart illustrating an operation of the ear-attaching type device 1 according to the second interval setting process. The second interval setting process is a process realized with the CPU 100 executing the second interval setting program 226 stored in the ROM 102, and it is executed in Step E36 of the second pulse measuring process.

First, the CPU 100 assigns a value obtained by subtracting the upper limit of the set range from the measured pulse rate as a variable X (Step G10). Next, if X is not more than a threshold of an item B stored in the reporting range setting data 320 (Step G12; Yes), the CPU reads out the time tc from the pitch time table 220 (Step G14). Then, the CPU 100 sets the time tc to the pitch interval data 324 (Step G16).

On the contrary, if X is more than the threshold of the item B stored in the reporting range setting data 320 (Step G12; No), the CPU 100 judges whether X is not more than a threshold of an item A stored in the reporting range setting data 320 (Step G18). Then, if X is not more than the threshold of the item A stored in the reporting range setting data 320 (Step G18; Yes), the CPU 100 reads out the time td from the pitch time table 220 (Step G20). Then, the CPU 100 sets the time td to the pitch interval data 324 (Step G22).

Further, if X is more than the threshold of the item A (Step G18; No), the CPU 100 reads out the time t0 from the pitch time table 220 (Step G24). Then, the CPU 100 sets the time t0 to the pitch interval data 324 (Step G26).

[2-3 Operation Example]

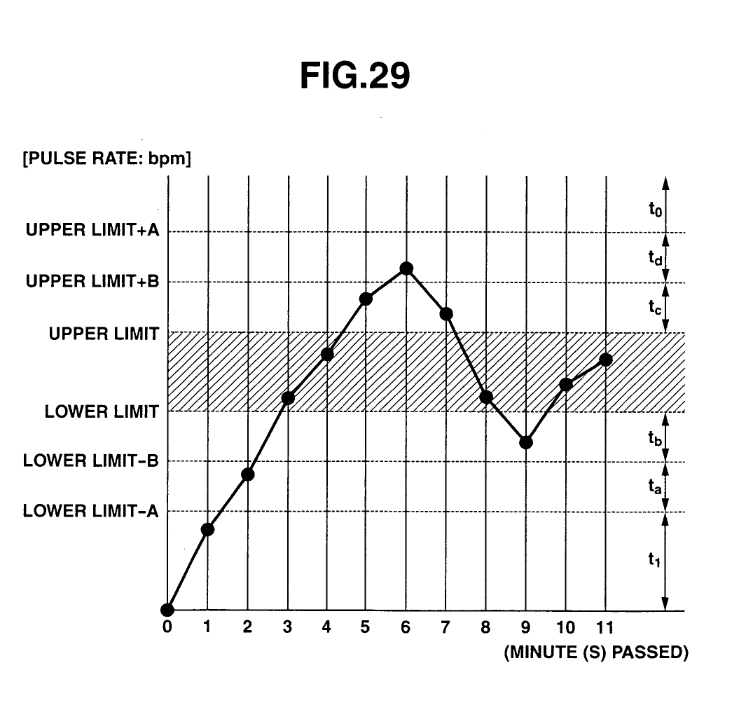

Here, the operation will be described concretely with reference to FIG. 29. FIG. 29 is a graph showing a transition of a pulse rate, with horizontal axis showing time (unit: minute) and vertical axis showing measured pulse rate (unit: bpm: beats per minute). Further, a meshed part is space between the upper limit “131” bpm and the lower limit “105” bpm of the set range stored in the set range data 308.

First, measured pulse rates at “1 MINUTE PASSED” and “2 MINUTES PASSED” are below the set range and there is no former pulse rate being within the set range (Step E30; Yes→Step E34; No). therefore, by setting infinite time to the pitch interval data 324 (Step E35), the CPU 100 does not output pitch sound (Step E58). Next, a measured pulse rate at “3 MINUTES PASSED” is within the set range, and the former pulse rate at “2 MINUTES PASSED” is not within the set range (Step E30; No→Step E32; No→Step E40; No) Then, since the timer is not in operation (Step E52; No), the CPU 100 sets a value of the measured pitch data 322 to the pitch interval data 324 (Step E56), and outputs reporting sound based on the set value (Step E58).

Next, a measured pulse rate at “4 MINUTES PASSED” is within the set range, and the former pulse rate at “3 MINUTES PASSED” is also within the set range (Step E30; No→Step E32; No→Step E40; Yes). Then, since the timer is not in operation (Step E42; No), the CPU 100 starts the timer (Step E44). Next, since the predetermined time “2 minutes” has not passed (Step E46; No), the CPU 100 outputs pitch sound based on the pitch interval data 324 (Step E58).

Next, a measured pulse rate at “5 MINUTES PASSED” is above the upper limit of the set range (Step E30; No→Step E32; Yes). Therefore, the CPU 100 executes the second interval setting process. Here, since a differential from the measured pulse rate at “5 MINUTES PASSED” to the upper limit of the set range data 308 is not more than the threshold of the item B (Step G12; Yes), the CPU 100 reads out the time tc “700 msec” from the pitch time table 220 (Step G14), and sets the time tc to the pitch interval data 324 (Step G16). Then, the CPU 100 outputs pitch sound according to an interval of the time “700 msec” set in the pitch interval data 324 (Step E58).

Next, a measured pulse rate at “6 MINUTES PASSED” is also above the upper limit of the set range (Step E30; No→Step E32; Yes). Therefore, the CPU 100 executes the second interval setting process. Here, since a differential from the measured pulse rate at “6 MINUTES PASSED” to the upper limit of the set range data 308 is more than the threshold of the item B and not more than the threshold of the item A (Step G12; No→Step G18; Yes), the CPU 100 reads out the time td “750 msec” from the pitch time table 220 (Step G20) and sets the time td to the pitch interval data 324 (Step G22). Then, the CPU 100 outputs pitch sound according to an interval of the time “750 msec” set in the pitch interval data 324 (Step E58).

Then, since a measured pulse rate at “8 MINUTES PASSED” is within the set range (Step E30; No→Step E40; No), the CPU 100 judges whether the timer is in operation. Here, since the timer has been in operation since “4 MINUTES PASSED” (Step E52; Yes), the CPU 100 stops and resets the timer (Step E54). Then, the CPU 100 reads out a calculated pitch interval from the measured pitch data 322, and sets the read-out data to the pitch interval data 324 (Step E56). Then, the CPU 100 outputs pitch sound based on the pitch interval data 324 (Step E58).

Further, since a differential from a measured pulse rate at “9 MINUTES PASSED” to the lower limit of the set range data 308 is not more than the threshold of the item B (Step F12; Yes), the CPU 100 reads out the time tb “500 msec” from the pitch time table 220 (Step F14) and sets the time tb to the pitch interval data 324 (Step F16). Then, the CPU 100 outputs pitch sound according to an interval of the time “500 msec” set in the pitch interval data 324 (Step E58).

In this way, according to the second embodiment, by only attaching the ear-attaching type device, it is possible to measure pulse and further to output pitch sound based on a measured pulse rate so as to achieve appropriate exercise corresponding to an exercise purpose.

### THIRD EMBODIMENT

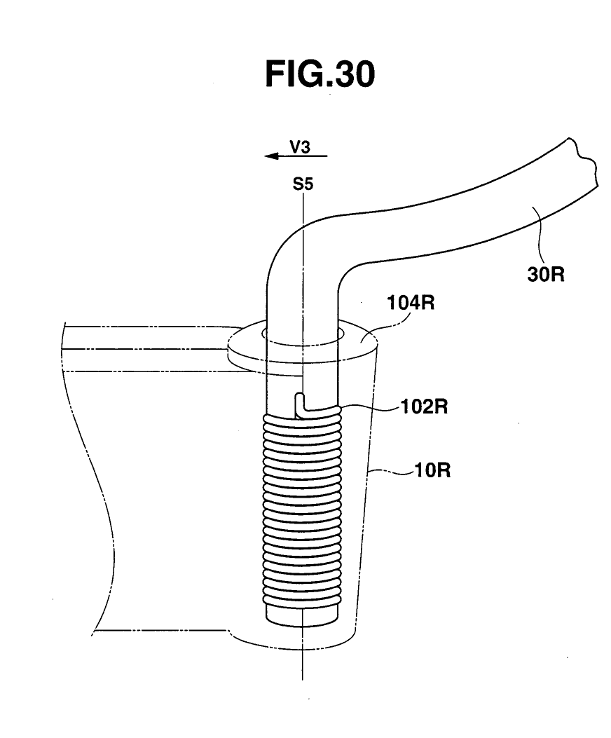

With reference to FIG. 30, an ear-attaching type device 1 in the third embodiment will be described in detail.

Here, FIG. 30 is a magnified view showing a right arm supporting member 10R for describing a biasing mechanism in the ear-attaching type device in the third embodiment of the present invention.

Here, in the third embodiment, while description regarding the biasing mechanism in the ear-attaching type device 1 is made, since everything other than the biasing mechanism is the same as the first embodiment, the description thereof is omitted.

In the ear-attaching type device in the third embodiment, the right arm part 3R is supported by the biasing mechanism in the body part 10 so as to bias the right arm part 3R at the right upper edge part of the body part 10 in the internal direction, and the left arm part 3L is supported so as to bias the left arm part 3L at the left upper edge part of the body part 10 in the internal direction (arrow V3).

In other words, the right arm supporting member 10R comprises a biasing mechanism such as a torsion coil spring 102R or the like for biasing the right arm part 3R in the direction of the arrow V3 (internal direction). As shown in FIG. 30, the biasing mechanism is structured so that the torsion coil spring 102 is wound into one edge of the right arm 30R inserted from an upper edge part 104R of the right arm supporting member 10R (this one edge part is more suitable in a cylindrical shape for transmitting elasticity of the spring) for biasing the right arm part 3R in the direction of the arrow V3. With such a biasing mechanism, the right arm part 3R is biased in the direction of the arrow V3 and rotated with respect to the rotation shaft S5.

Here, since the left arm supporting member 10L has approximately the same structure as the right arm supporting member 10R, the description thereof is omitted.

With the above-described structure, in order to attach the ear-attaching type device, a user holds the right arm part 3R and the left arm part 3L and widens them to a direction in which the right driver unit 34R and the left driver unit 34L separate from each other. Then, the user moves the ear-attaching type device 1 so as to go around the head part from the occipital part H Side, and the user attaches the ear-attaching type device 1 with himself/herself by inserting the right driver unit 34R into an ear hole 7R of the right ear and the left driver unit 34L into an ear hole 7L of the left ear.

At this time, according to a bias transmitted to the right driver unit 34R and the left driver unit 34L through the right arm part 3R and the left arm part 3L, the right driver unit 34R and the left driver unit 34L are biased toward a direction of inside of the ear holes (internal direction). Further, as shown in FIG. 1B, since a back surface of the body part 10 (rear surface of the operation panel 16) is contacted with a lower part of the occipital part H, the posture of the body part 10 is maintained.

As above, according to the present embodiment, the right arm part 3R is biased in the internal direction with the biasing mechanism of the right arm supporting member 10R, and the left arm part 3L is biased in the internal direction with the biasing mechanism of the left arm supporting member 10L (in the direction of the arrow V3). Thereby, a bias by the biasing mechanism is transmitted through the right arm part 3R to the right driver unit 34R, and is transmitted through the left arm part 3L to the left driver unit 34L. Then, each of the driver units 34R and 34L is biased toward inside of the ear holes by being biased toward the center of the head part, that is, in a direction in which each driver unit comes close. According to this bias in the internal direction in addition to each of the driver units 34R and 34L being in a half sphere shape, the driver unit is not easily fallen off from an ear hole and is not easily misaligned despite the movement of user's head, whereby it is possible to obtain a sense of stable attachment even during exercise.

Further, considering the fact that the pivot shaft S1 connecting the right driver unit 34R and the left driver unit 34L (see FIG. 1A) is a rotation shaft of the ear-attaching type device, since the body part 10 having a certain weight by incorporating therein a battery or the like is stabilized in a state of contacting with a lower part of the occipital part H according to its own weight, the posture of the ear-attaching type device is maintained. Therefore, according to the own weight of the body part 10, a fluctuation in a rotation direction with respect to the pivot shaft S1 by the head movement is also suppressed, and thereby it is possible to obtain a sense of stable attachment.

Further, since there are three contacting surfaces to the head part, which are the right driver unit 34R, the left driver unit 34L and the body part 10, surrounding sense is reduced compared to an ear-sealing type headphone, whereby it is possible to obtain a more comfortable sense of attachment.

Further, the right arm part 3R is contacted with the head part according to the bias from the right side of the ear 11R (illustration omitted), and the left arm part 3L is contacted with the head part according to the bias from the outside of the ear 11L. Therefore, since the right arm part 3R does not use the base part of the ear 11R and the left arm part 3L does not use the base part of the ear 11L, it is possible to wear a pair of glasses while attaching the ear-attaching type device.

Further, since the pulse sensor unit 5 is pinched to be engaged with the earlobe 9L of the left ear, a part regarding the attachment of the ear-attaching type device is only a head part. Therefore, for example, when a user swings his/her arm up at the time of jogging, he/she is not bothered any more with the cable 50 disturbing the arm swinging. Accordingly, it is possible to reduce the botheration of the cable 50 while the ear-attaching type device is being attached.

Further, with the pulse sensor unit 5 connected to the left side surface of the body part 10, a user pinches the pulse sensor unit 5 to the earlobe 9L of the left ear. The pulse switch 36R for listening to a pulse rate of the pulse detected by the pulse sensor unit 5 is placed on the right driver unit supporting member 32R, which is at the opposite side of the earlobe 9L to which the pulse sensor unit 5 is attached. Therefore, when a user performs a pushing operation of the pulse switch 36R for listening to a pulse rate, a user is kept from touching the pulse sensor unit 5 by mistake, whereby it is possible to reduce a negative effect on the pulse detection.

Further, according to the present embodiment, since a rotation mechanism portion for rotating the arm part with respect to the body part and a rotation stopping mechanism portion for stopping the rotation of the arm part by the rotation mechanism portion are provided, it is possible that the rotation stopping mechanism portion stops the rotation of the arm part with respect to the body part by the rotation mechanism portion. Thereby, even if the arm part is rotated, unreasonable force is not applied to the connecting member placed inside of the arm part, and it is possible to prevent from breaking the connecting member.

Further, by stopping the arm part at the attaching position and the housing position of the ear-attaching type device with the rotation stopping mechanism portion, it is possible to attach and house the ear-attaching type device easily.

Further, the rotation stopping mechanism portion comprises an attaching position stopping portion for stopping the rotation of the arm part at the attaching position of the ear-attaching type device, and a housing position stopping portion for stopping the rotation of the arm part at the device housing position of the ear-attaching type device. Therefore, it is possible to determine a rotation stopping position of the arm part with the device position stopping portion while the ear-attaching type device is being attached. Further, it is possible to determine a rotation stopping position of the arm part with the housing position stopping portion at the time of housing the ear-attaching type device. Moreover, one of the first body case member and the second body case member comprises the attaching position stopping portion and another comprises the housing position stopping portion, it is possible to have more variance of a position where one of the attaching position stopping portion and the housing position stopping portion is placed than a case of placing both of the attaching position stopping portion and the housing position stopping portion in one of the first body case member and the second body case member. Thereby, it is possible to simplify the structures of the first body case member and the second body case member. Accordingly, it is possible to easily form the first body case member and the second body case member.

Further, since the rotation mechanism portion comprises a sliding guide for guiding the rotation of the arm part so as to slide the external surface of the arm part against the internal surface of the body part, it is possible to guide the rotation of the arm part by the sliding guide with respect to the body part, whereby it is possible to rotate the arm part more properly.

Further, since a groove to be engaged with a rib protruding from the internal surface of the inside of the body part is formed along the sliding direction, it is possible to guide the rotation of the arm part more properly, whereby it is possible to suppress tilt, irregular movement and the like of the arm part.

Further, the sliding guide is provided in a shaft member having a rotation shaft of the arm part, and has a rotation stopping surface for stopping the rotation of the arm part by the rotation stopping mechanism portion the rotation stopping surface extending in a radial direction from the rotation shaft, wherein an internal surface thereof is formed in approximately an arc shape. Therefore, it is possible to guide the rotation of the arm part properly so as to slide the internal surface, and it is possible to stop the rotation of the arm part properly by the rotation stopping surface.

Further, the rotation mechanism portion comprises the shaft member having the rotation shaft of the arm part, wherein the shaft member comprises a flange member which becomes thicker as coming close to the rotation shaft. Therefore, it is possible to intensify the strength around the rotation shaft at the side of the rotation shaft of the flange member, whereby it is possible to rotate the arm part more properly.

Further, since the internal surface of the flange member is formed so as to dent toward the side of the rotation shaft, it is possible to more properly secure an implementation range of devices placed inside of the body part. Further, since the internal surface of the flange member is formed in a curved shape so as to follow the internal wall of the body part, it is possible to have a large contacting area of the internal surface of the flange member with the internal wall. Therefore, it is possible to guide the rotation of the arm part more suitably, whereby it is possible to properly suppress tilt, irregular movement and the like of the arm part.

### MODIFIED EXAMPLE

So far, what is described in the present embodiments is the ear-attaching type device with a pulse measuring function as an applied example. However, what the present invention can be applied to is not limited to such a product, and various modifications and design changes may be suitably done without departing the gist of the present invention.

For example, other than a pulse rate, body temperature or blood pressure may be measured. Further, described is the case that the ear-attaching type device comprises a radio function. However, the ear-attaching type device may comprise a music playing device, or various electronic devices such as a cellular phone and the like.

Further, values stored in various storing areas and tables in the present embodiments are one example, and, needless to say, it is possible to change the stored values. Further, the description is made by illustrating the case that a user inputs a pulse rate at resting to be stored in the individual data 306. However, a pulse rate at the time that a user is resting may be in reality measured to be set as the pulse rate at resting. If a pulse rate at resting is set in this way, it is possible to set the pulse rate at resting of a user easily.

Further, in the present embodiments, described is the case that the reporting is made by giving advice with sound and outputting pitch sound. However, elapsed exercise time or remaining exercise time may be reported. Concretely, if 10 minutes have passed since the measurement of pulse, the sound “10 minutes elapsed” is automatically outputted from the sound outputting unit 116. By doing such report, a user can recognize elapsed exercise time or remaining exercise time appropriately.

Further, in the present embodiments, described is the case that the pulse sensor unit 5 is connected to the left side of the ear-attaching type device 1, the pulse switch 36R is placed on the right driver unit supporting member 32R and the tuner switch 36L is placed on the left driver unit supporting member 32L. However, the present invention is not limited to such a case. Needless to say, the pulse sensor unit 5 may be connected to the right side, and the pulse switch 36R and the tuner switch 36L may be placed in the opposite way.

Further, described is the case that a driver unit is formed in the so-called vertical type, in which the sound emitting surface 72 faces in the front direction. However, a driver unit may be formed in a sealed-up type in which the sound emitting surface faces in a direction toward the inside of the head part, or may be formed in an open-air type which leaves surrounding sense at a certain degree.

Further, described is the case that pulse rate is measured and outputted with sound. However, for example, the pulse sensor unit may optically detect oxygen saturation in blood and output it with sound, or an electronic thermometer may be incorporated in the driver unit for measuring body temperature within an ear hole to output it with sound.

Further, described is the case that the right arm supporting member 10R and the left arm supporting member 10L respectively support each of the arm parts 3R and 3L so as to bias each arm part in the internal direction. However, the present invention is not limited to such a case, and a suitable setting change may be applied.

For example, as shown in FIG, 31A, a headband may be formed from material having flexibility and elasticity such as polypropylene, the headband being in approximately a shape of letter ‘U’ when it is seen from the top view, wherein the right driver unit 34R and the left driver unit 34L are respectively supported at the edges thereof. By flexing the headband 200 to be attached, a bias (elastic force) is caused in the internal direction of the headband 200. Therefore, as well as the case of the present embodiment, it is possible to bias each driver unit toward ear holes.

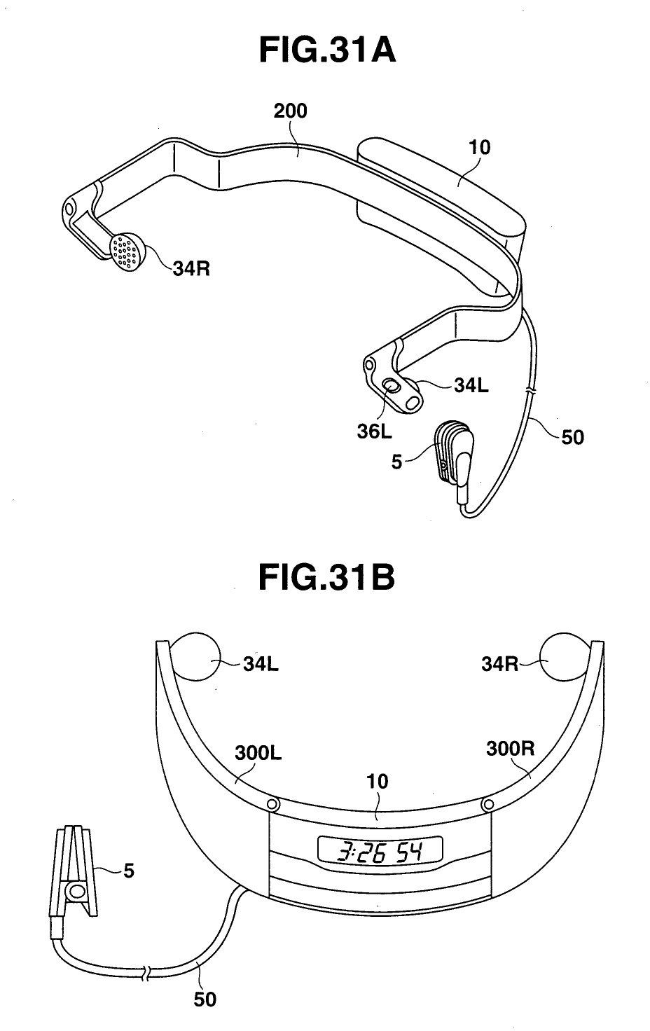

Further, for example, as shown in FIG. 31B, a hinge member may be provided at both the edges of the body part 10, for forming a biasing mechanism such as a torsion coil spring at a part of the hinge member. Then, the ear-attaching type device may be structured so that a right band 300R and a left band 300L extend from the body part 10.

1. An ear-attaching type electronic device comprising:
a body part which is supported in a vicinity of a lower part of an occipital part when the device is attached;
a pair of arm parts extending from the body part;
a pair of speakers respectively supported at edge parts of the arm parts;
a detecting section fixed to one of a left earlobe and a right earlobe, for detecting biological information and for outputting the detected biological information to the body part;
a sound outputting section for outputting a sound signal to the pair of speakers; and
a measuring section for measuring the biological information outputted from the detecting section.

2. The device according to claim 1, wherein
the body part comprises a biasing mechanism for biasing each arm part in a direction in which each of the edge parts comes close, and
the biasing mechanism determines a posture of the device when the device is attached, by establishing a pivot shaft which is a line passing through left and right ears, according to a bias by the biasing mechanism transmitted to each of the speakers through each of the arm parts.

3. The device according to claim 1, wherein a vibration detecting section is incorporated in the body part, and
the vibration detecting section measures walking pitch or jogging pitch.

4. An ear-attaching type electronic device comprising:
a body part supported in a vicinity of a lower part of an occipital part when the device is attached;
a pair of arm parts extending from the body part;
a pair of speakers respectively supported at edge parts of the arm parts;
a detecting section fixed to one of a left earlobe and a right earlobe, for detecting biological information and for outputting the detected biological information to the body part; and
an operation instructing section placed at a side of an arm part corresponding to another one of the left earlobe and the right earlobe, for performing an operation instruction regarding a measurement of the biological information.

5. An ear-attaching type electronic device comprising:
a body part supported in a vicinity of a lower part of an occipital part when the device is attached;
a pair of arm parts extending from the body part;
a pair of speakers respectively supported at edge parts of the arm parts;
a detecting section fixed to one of a left earlobe and a right earlobe, for detecting pulse and for outputting a wave form of the detected pulse to the body part;
a sound outputting section for outputting a sound signal to the pair of speakers;
a measuring section for measuring the wave form of the pulse outputted from the detecting section; and
an operation instructing section placed at a side of an arm part corresponding to another one of the left earlobe and the right earlobe, for performing an operation instruction regarding a measurement of the biological information.

6. An ear-attaching type electronic device comprising:
a body part supported in a vicinity of a lower part of an occipital part when the device is attached;
a pair of arm parts extending from the body part;
a pair of speakers respectively supported at edge parts of the arm parts;
a sound outputting section placed in the body part, for outputting a sound signal to the pair of speakers;
a connecting member placed inside of each of the arm parts, for electrically connecting the sound outputting unit and each of the speakers;
a rotation mechanism portion for rotating each of the arm parts with respect to the body part; and
a rotation stopping mechanism portion for stopping a rotation of each of the arm parts by the rotation mechanism portion.

7. The device according to claim 6, wherein the rotation stopping mechanism portion stops the rotation of each of the arm parts at a device attaching position and a device housing position.

8. The device according to claim 7, further comprising:
a device position stopping portion for stopping the rotation of each of the arm parts at the device attaching position;
a housing position stopping portion for stopping the rotation of each of the arm parts at the device housing position; and
a first body case member and a second body case member each of which is formed in a convex shape so as to make an opening side thereof face each other,
wherein the attaching position stopping portion is provided in one of the first body case member and the second body case member and the housing position stopping member is provided in another one of the first body case member and the second body case member.

9. The device according to claim 6, wherein the rotation mechanism portion comprises a sliding guide for guiding the rotation of each of the arm parts so as to slide an external surface against an internal surface of the body part.

10. The device according to claim 9, wherein
a rib which protrudes from the internal surface, is provided inside of the body part, and
a groove which is engaged with the rib, is formed along a sliding direction on an external surface of the sliding guide.

11. The device according to claim 9, wherein the rotation mechanism portion comprises a shaft member which has a rotation shaft of each of the arm parts at a position inside of the body part by being attached to the body part of the arm part, and
the sliding guide is provided in the shaft member, has an external surface formed in approximately an arc shape, extends in a radial direction from the rotation shaft, and comprises a rotation stopping surface for stopping the rotation of each of the arm parts by using the rotation stopping mechanism portion.

12. The device according to claim 6, wherein the rotation mechanism portion comprises a shaft member which has a rotation shaft of the arm parts at a position inside of the body part by being attached to the body part of the arm part, and
the shaft member comprises a flange member which is formed so that the flange member becomes thicker as coming close to the rotation shaft.

13. The device according to claim 12, wherein the flange member comprises an external surface which is formed in a curved shape so as to dent toward a side of the rotation shaft and to follow an internal wall of the body part.

14. An ear-attaching type electronic device comprising:
a body part supported in a vicinity of a lower part of an occipital part when the device is attached;
a pair of arm parts extending from the body part;
a pair of speakers respectively supported at edge parts of the arm parts;
a detecting section fixed to one of a left earlobe and a right earlobe, for detecting biological information and for outputting the detected biological information to the body part;
a calculating section for calculating a bloodstream state value indicating a bloodstream state according to the biological information detected by the detecting section;
a range setting section for previously setting a range of a bloodstream state value to be targeted;
a comparing section for comparing the range of the bloodstream state value set by the range setting section with the bloodstream state value calculated by the calculating section; and
a reporting section for reporting advice corresponding to a comparison result by the comparing section, with sound.

15. The device according to claim 14, wherein
the detecting section for detecting the biological information is provided at a position being appropriate to be fixed to one of a left earlobe and a right earlobe, and
an operation button for performing an operation with respect to calculation of the bloodstream state value is provided at a supporting member of a speaker corresponding to another one of the left earlobe and the right earlobe.

16. The device according to claim 14, further comprising:
a sound outputting section for outputting sound; and
a volume controlling section for controlling the sound outputting section so that, when the reporting section reports the advice while the sound outputting section is outputting the sound, an output volume of the sound being outputted by the sound outputting section is temporarily lowered down to let the reporting section report the advice.

17. The device according to claim 14, further comprising:
a vibration detecting section;
a pitch measuring section for measuring pitch of walking or jogging according to vibration detected by the vibration detecting section; and
a pitch sound adjustment outputting section for adjusting pitch sound corresponding to the pitch measured by the pitch measuring section according to the comparison result by the comparing section to be outputted.

18. The device according to claim 14, further comprising:
a vibration detecting section;
a pitch measuring section for measuring pitch of walking or jogging according to vibration detected by the vibration detecting section;
a pitch sound adjustment outputting section for adjusting pitch sound corresponding to the pitch measured by the pitch measuring section according to the comparison result by the comparing section to be outputted; and
a pitch sound interval adjusting section for controlling an interval of pitch sound so that the interval of the pitch sound is shortened when the comparing section judges that the bloodstream state value calculated by the calculating section is less than the range of the bloodstream state value set by the range setting section, and the interval of the pitch,sound is widened when the comparing section judges that the bloodstream state value calculated by the calculating section is more than the range of the bloodstream state value set by the range setting section

19. A biological information measuring method in an ear-attaching type electronic device which comprises a body part supported in a vicinity of a lower part of an occipital part when the device is attached; a pair of arm parts extending from the body part; and a pair of speakers respectively supported at edge parts of the arm parts, comprising:
detecting biological information of one of a right earlobe and a left earlobe;
calculating a bloodstream state value indicating a bloodstream state according to the detected biological information;
setting previously a range of a bloodstream state value to be targeted
comparing the set range of the bloodstream with the calculated bloodstream state value; and
reporting advice corresponding to a result of the comparing, with sound.

20. A biological information measuring method in an ear-attaching type electronic device which comprises a body part supported in a vicinity of a lower part of an occipital part when the device is attached; a pair of arm parts extending from the body part; and a pair of speakers respectively supported at edge parts of the arm parts, comprising:
detecting biological information of one of a right earlobe and a left earlobe;
calculating a bloodstream state value indicating a bloodstream state according to the detected biological information;
setting preliminarily a range of a bloodstream state value to be targeted
comparing the set range of the bloodstream with the calculated bloodstream state value; and
reporting pitch sound when the calculated bloodstream state value is not included in the set range, the pitch sound corresponding to the calculated bloodstream state value.
